(19) United States
(12) Patent Application Publication
(10) Pub. No.: US 2016/0280742 A1
(43) Pub. Date: Sep. 29, 2016

(54) Compositions and Methods for Treating Alzheimer's Disease

(71) Applicants: The University of British Columbia, Vancouver (CA); CNJ Holdings Inc., Winnipeg (CA)

(72) Inventors: Neil R. Cashman, Vancouver (CA); Grant McClarty, Winnipeg (CA)

(21) Appl. No.: 15/056,546

(22) Filed: Feb. 29, 2016

Related U.S. Application Data
(63) Continuation of application No. 14/357,399, filed on May 9, 2014, now abandoned.
(60) Provisional application No. 61/589,809, filed on Jan. 23, 2012.
(60) Provisional application No. 61/558,430, filed on Nov. 10, 2011.

(51) Int. Cl. C07K 7/64; A61K 39/395; C07K 16/18; C07K 14/47; G01N 33/68

(52) U.S. Cl. CPC C07K 7/64; C07K 14/4711; G01N 33/6896; C07K 16/18; A61K 39/3955; A61K 39/00

(57) ABSTRACT

The present disclosure provides for a hyperimmune preparation comprising human polyclonal antibodies or fragments thereof specific to a cyclic peptide having an amino acid sequence comprising SNK. The cyclic peptide may comprise an amino acid sequence GSNK (SEQ ID NO: 1), SNKG (SEQ ID NO: 2), GSNKG (SEQ ID NO: 3), CSNKG (SEQ ID NO: 4), CGSNKGC (SEQ ID NO: 5), CGSNKGG (SEQ ID NO: 6), or CCGSNKGC (SEQ ID NO: 7). The antibodies in the hyperimmune preparation may have a titer ranging from about 200 to about 400 mean fluorescence intensity (MFI). In one embodiment, greater than about 80% of the antibodies in the hyperimmune preparation are IgG. The fragments of the antibodies are Fab, F(ab′)2, scFv, disulfide linked Fv, or mixtures thereof.

### CROSS REFERENCE TO RELATED APPLICATIONS

This application claims priority to U.S. Provisional Application No. 61/558,430 (filed on Nov. 10, 2011) and 61/589,809 (filed on Jan. 23, 2012), and U.S. application Ser. No. 13/582,308 (filed on Aug. 31, 2012), each of which is incorporated herein by reference in its entirety.

### FIELD

The present invention relates to compositions and methods for the treatment and prophylaxis of Alzheimer's disease in a subject. The invention also discloses compositions and methods for screening and diagnosing Alzheimer's disease.

### BACKGROUND

Alzheimer's disease (AD) is a common dementing (disordered memory and cognition) neurodegenerative disease. It is associated with accumulation in the brain of extracellular plaques composed predominantly of the amyloid beta peptides (also referred to as amyloid β, Abeta or Aβ), including Aβ (1-40), Aβ (1-42) and Aβ (1-43) peptides. These Aβ peptides are proteolytic products of amyloid precursor protein (APP). In addition, neurofibrillary tangles, composed principally of abnormally phosphorylated tau protein (a neuronal microtubule-associated protein), accumulate intracellularly in dying neurons. The Aβ (1-42) is the dominant species in the amyloid plaques of Alzheimer's disease patients. Aβ oligomerization has been shown to be a key part of neurotoxicity in Alzheimer's disease.

It has been found that a particular molecular species of Aβ, in which the peptide is oligomerized, mediates the major component of neurotoxicity observed in Alzheimer's disease and mouse models of the disease (Walsh et al., Naturally secreted oligomers of amyloid beta protein potently inhibit hippocampal long-term potentiation in vivo, Nature, 2002, 416(6880): 535-9). Aβ oligomer toxicity can be manifested by dysfunction of neuronal insulin receptors (Zhao et al., Amyloid beta oligomers induce impairment of neuronal insulin receptors, FASEB J. 2008, 22(1):246-60), and by interference with normal synaptic function, particularly in the hippocampus, by ectopic activation of glutamatergic receptors (De Felice et al., 2007. Abeta oligomers induce neuronal oxidative stress through an N-methyl-D-aspartate receptor-dependent mechanism that is blocked by the Alzheimer drug memantine, J. Biol. Chem. 282:11590-11601; Nimmrich et al., Amyloid beta oligomers (Abeta(1-42) globulomer) suppress spontaneous synaptic activity by inhibition of P/Q-type calcium currents, J. Neurosci., 2008, 23; 28(4):788-97). Amyloid plaques are generally formed via aberrant cleavage of the amyloid precursor protein (APP) by beta-secretase and then gamma-secretase, to form the insoluble peptide Aβ (1-42). These peptides aggregate to form the amyloid plaques that are one of the hallmarks of Alzheimer's disease. Although a large number of cerebral amyloid plaques are usually associated with Alzheimer's disease, cognitive loss has been found to correlate poorly with the number of amyloid plaques. Instead, cognitive loss has been found to better correlate with other forms of Aβ, for example soluble Aβ oligomers or aggregates, suggesting that Aβ oligomers might be more directly linked to neuronal and synaptic loss.

Many in vitro and in vivo studies have been conducted and the results demonstrate that immune therapy against Aβ can lead to the improvement of both the pathology and behavior of transgenic mice expressing human mutant APP. Unfortunately, these positive immunotherapy results in mice have not translated well in humans as there were adverse events associated with the treatment, including autoimmune meningoencephalitis, during clinical trials (Gilman et al., Clinical effects of Abeta immunization (AN1792) in patients with AD in an interrupted trial, Neurology, 2005, 10; 64(9):1553-62). However, there are still numerous immunotherapy treatments, both passive and active, in clinical trials.

In healthy individuals, antibodies specific to at least Aβ(1-42) are naturally present. It was reported that the concentration of antibodies against the oligomeric forms of Aβ(1-42) in particular declined with age and advanced Alzheimer's disease. Britschgi et al., Neuroprotective natural antibodies to assemblies of amyloidogenic peptides decrease with normal aging and advancing Alzheimer's disease. Proc. Natl. Acad. Sci. USA., 2009, 106(29):12145-50.

It is desirable to develop biologics that arrest or slow down the progression of the disease without inducing negative and potentially lethal effects on the human body. The need is particularly evident in view of the increasing longevity of the general population and, with this increase, an associated rise in the number of patients annually diagnosed with Alzheimer's disease. It is also desirable to develop diagnostic tools for determining the various stages of disease progression.

### SUMMARY

The present disclosure provides for a hyperimmune preparation comprising human polyclonal antibodies or fragments thereof specific to a cyclic peptide having an amino acid sequence comprising SNK. The cyclic peptide may comprise an amino acid sequence GSNK (SEQ ID NO: 1), SNKG(SEQ ID NO: 2), GSNKG (SEQ ID NO:3), CSNKG (SEQ ID NO: 4), CGSNKGC (SEQ ID NO: 5), CGSNKGG (SEQ ID NO: 6), or CCGSNKGC (SEQ ID NO: 7). The antibodies in the hyperimmune preparation may have a titer ranging from about 200 to about 400 mean fluorescence intensity (MFI). In one embodiment, greater than about 80% of the antibodies in the hyperimmune preparation are IgG. The fragments of the antibodies are Fab, F(ab′)₂, scFv, disulfide linked Fv, or mixtures thereof.

The hyperimmune preparation can be prepared from human plasma or serum. In one embodiment, the hyperimmune preparation is intravenous immunoglobulin (IVIG).

The hyperimmune preparation may be in a form suitable for parenteral injection or infusion.

The present disclosure also provides for a method for treatment or prophylaxis of Alzheimer's disease in a subject comprising the step of administering to the subject a pharmaceutically effective amount of a hyperimmune preparation comprising human polyclonal antibodies or fragments thereof specific to a cyclic peptide having an amino acid sequence comprising SNK. The cyclic peptide may comprise an amino acid sequence GSNK(SEQ ID NO: 1), SNKG(SEQ ID NO: 2), GSNKG (SEQ ID NO:3), CSNKG (SEQ ID NO: 4), CGSNKGC (SEQ ID NO: 5), CGSNKGG (SEQ ID NO: 6), or CCGSNKGC (SEQ ID NO: 7). The hyperimmune preparation may be administered by parenteral injection or infusion, such as intravenous injection or infusion. The hyperimmune preparation may be administered at a dose ranging from about 10 μg to about 200 mg antibodies per kg body weight, or from about 10 μg to about 400 μg antibodies per kg body weight. The hyperimmune preparation can also be administered subcutaneously, intramuscularly, transdermally or orally.

The hyperimmune preparation may be administered consecutively, simultaneously or in combination with an acetylcholinesterase inhibitor (such as tacrine, rivastigmine, galantaminc and donepezil) or an NMDA receptor antagonist (such as memantine).

Also encompassed by the present disclosure is a method of diagnosing Alzheimer's Disease in a subject comprising the steps of: (a) obtaining a biological sample from the subject; (b) quantifying in the sample the level of antibodies specific to a conformational epitope having an amino acid sequence comprising SNK; and (c) comparing the level of the antibodies in step (b) with a control sample.

The present disclosure provides for a method of predicting a subject's risk of developing Alzheimer's Disease comprising the steps of: (a) obtaining a biological sample From the subject; (b) quantifying in the sample the level of antibodies specific to a conformational epitope having an amino acid sequence comprising SNK; and (c) comparing the level of the antibodies in step (b) with a control sample.

The biological sample may be plasma, tissues, cells, biofluids (e.g., cerebrospinal fluid (CSF) or blood) or combinations thereof. The control sample may be a sample from one or more Alzheimer's Disease-free subjects; or a sample from the same subject obtained a time period ago wherein the time period ranges from about 6 months to about 5 years.

### BRIEF DESCRIPTION OF THE DRAWINGS

FIG. 1 shows MagPlex® immunoassay (based on the Luminex xMAP technology) results graphically illustrating the level of specific antibody binding as mean fluorescence intensity (MFI) for the cyclic peptide derived from Aβ, within a population of plasma donors.

FIG. 2 shows MagPlex® immunoassay (based on the Luminex xMAP technology) results graphically illustrating the level of specific antibody binding as mean fluorescence intensity (MFI) for the cyclic peptide derived from Aβ, Aβ (1-42), and Aβ (1-40) front the 10 highest titer donors to the cyclic peptide derived from Aβ within a population of plasma donors.

FIG. 3 shows MagPlex® immunoassay (based on the Luminex xMAP technology) results graphically illustrating the percentage of plasma donors having specific antibody binding above background (mean fluorescence intensity (MFI)>1) for Aβ protein (1-42), Aβ protein (1-40), the cyclic peptide derived from Aβ, and the percentage of plasma donors with no binding (mean fluorescence intensity (MFI)<1).

FIG. 4 shows MagPlex® immunoassay (based on the Luminex xMAP technology) results graphically illustrating the percentage of plasma donors positive for antibodies (mean fluorescence intensity (MFI)>2 times the mean fluorescence intensity of both other peptides tested) to the cyclic peptide derived from Aβ and Aβ (1-42) protein categorized by the age range of donors.

FIG. 5 shows MagPlex® immunoassay (based on the Luminex xMAP technology) results graphically illustrating the age distribution of donors with high titer of specific antibodies (mean fluorescence intensity (MFI)>50).

FIG. 6 shows MagPlex® immunoassay (based on the Luminex xMAP technology) results graphically illustrating the percentage of male and female plasma donors positive for antibodies (mean fluorescence intensity >2 times the mean fluorescence intensity of both other peptides tested) to the cyclic peptide derived from Aβ and Aβ (1-42).

FIG. 7 is a three dimensional model of the CSNKG cyclic peptide.

FIG. 8 is a three dimensional model of the CGSNKGG cyclic peptide.

FIG. 9 is a three dimensional model of the acetylated-CCGSNKGC cyclic peptide.

FIGS. 10A-10G show MagPlex® immunoassay and ELISA results suggesting that antibodies to misfolded amyloid-β peptides are naturally occurring in normal adults. FIG. 10A: Prevalence of plasma donors with antibodies to amyloid-β peptides. FIG. 10B: Percentage of plasma donors with high levels of antibodies to SNK cyclic peptide. FIGS. 10C and 10D: younger population had higher levels of naturally occurring antibodies to misfolded amyloid-β epitope. FIG. 10E: Titers of misfolded Abeta epitope by ELISA. FIG. 10F: prevalence of female donors with antibodies to misfolded aβ epitope. FIG. 10G: prevalence of male donors with antibodies to misfolded aβ epitope.

FIGS. 11A and 11B. Representative sensorgrams showing the binding of purified IgG to Abeta(1-42) oligomer and cSNK peptide. Synthetic Abeta(1-42) oligomers and BSA-conjugated cSNK peptides were covalently immobilized on separate flow cells of a Biacore™ sensorchip and purified IgG sequentially injected over the flow cells. FIG. 11A: binding to immobilized Abeta(1-42) oligomer. FIG. 11B: binding to immobilized BSA-conjugated cSNK.

FIG. 12. Correlating the binding of purified IgG to Abeta(1-42) oligomer and cSNK peptide. Synthetic Abeta(1-42) oligomers and BSA-conjugated cSNK peptides were covalently immobilized on separate flow cells of a Biacore™ sensorchip and purified IgG sequentially injected over the flow cells. Resultant binding responses were then compared by Pearson Correlation.

FIG. 13. Purified IgG antibody profiling. The isotype/subclass of antibody bound to immobilized cSNK (a) was determined by the sequential injection of mouse anti-human IgG2 (b), IgG3 (c), IgG4 (d), total IgG (e), IgM (f) and IgG1 (g).

FIG. 14. Correlation between MagPlex™ and Biacore™ immunoassays for the binding of purified IgG to cSNK. BSA-conjugated cSNK peptides were coupled to Luminex® Superparamagnetic Carboxylated xMAP® Microspheres and to CM5 sensor chips, respectively, and the binding of purified IgG accordingly analyzed on a MAGPIX analyzer system and a Biacore™ 3000 biosensor. The binding responses were correlated (Pearson Correlation=0.708).

### DETAILED DESCRIPTION

The present invention provides for hyperimmune preparations, pharmaceutical compositions and methods for the treatment and prophylaxis of Alzheimer's disease. The hyperimmune preparations are enriched with antibodies specific to a conformational epitope of oligomeric Aβ. The antibodies in the hyperimmune preparations have a high titer. The conformational epitope corresponds to a solvent-exposed, antibody accessible knuckle region of oligomeric Aβ. The conformational epitope may be part of a cyclic peptide having an amino acid sequence comprising SNK (i.e., serine-asparagine-lysine, or Ser-Asn-Lys) in which the side chain of lysine is constrained to be oriented into solvent. In one embodiment, the conformational epitope may be part of a cyclic peptide having an amino acid sequence comprising at least SNK. The present compositions may be used for passive immunotherapy of Alzheimer's disease or prophylactic vaccines in populations at risk of Alzheimer's disease.

The present hyperimmune compositions are enriched with antibodies specific to a cyclic peptide having an amino acid sequence comprising SNK. The antibody titer may range from about 1 to about 1,000, about 5 to about 800, about 10 to about 600, about 1 to about 50, about 50 to about 100, about 50 to about 200, about 100 to about 200, about 200 to about 400, about 400 to about 600, about 600 to about 800, about 800 to about 1,000, about 1,000 to about 2,000, about 2,000 to about 3,000, about 3,000 to about 4,000, about 5,000 to about 10,000, or about 10,000 to about 20,000 mean fluorescence intensity (MFI). The MFI values correlate with the binding intensity on the MagPlex® immunoassay based on Luminex's xMAP® technology.

The antibody concentration or titer may range from about 100 μg/ml to about 5 mg/ml, about 200 μg/ml to about 4 mg/ml, about 500 μg/ml to about 3 mg/ml, about 1 mg/ml to about 2 mg/ml, about 2 mg/ml to about 3 mg/ml, about 3 mg/ml to about 4 mg/ml, about 4 mg/ml to about 5 mg/ml, about 100 μg/ml to about 200 μg/ml, about 200 μg/ml to about 300 μg/ml, about 300 μg/ml to about 400 μg/ml, about 400 μg/ml to about 500 μg/ml, about 500 μg/ml to about 600 μg/ml, about 600 μg/ml to about 800 μg/ml, or about 800 μg/ml to about 1 mg/ml.

The hyperimmune preparations may be prepared from human plasma from normal individuals or disease donors, such as those with Alzheimer's disease or one of the dementing family of diseases. The hyperimmune preparations may contain pooled immunoglobulins (i.e., antibodies). The present disclosure further provides a pharmaceutical composition comprising the hyperimmune preparation for passive immunotherapies.

Also encompassed by the present invention are compositions and methods for the diagnosis and screening of Alzheimer's disease.

When Aβ oligomerizes, a constrained peptide turn forms and takes on a knuckle-like conformation. In the knuckle region of oligomeric Aβ, the epitope GSNKG, including the lysine side chain, is exposed to solvent and accessible to antibody binding. This epitope represents a novel target in misfolded forms of Aβ. As used herein, the term “Aβ oligomer” or “oligomeric Aβ” refers to a form of the Aβ peptide where the Aβ monomers are non-covalently or covalently aggregated. An Aβ oligomer may have less than about 200 Aβ monomers. In one embodiment, an Aβ oligomer may have less than about 50 Aβ monomers.

Image capture of molecular dynamics modeling of a disulfide-linked cyclic peptide comprising residues 25-29 (CGSNKGC) was conducted; non-native cysteines were added for disulfide linkage. This modeling reveals that the side chain of lysine 28 is oriented externally as shown, in contrast to the internally oriented lysine 28 side chain predicted in references Luhrs et al., 3D structure of Alzheimer's amyloid-beta(1-42) fibrils, Proc. Natl. Acad. Sci. USA, 2005, 102(48): 17342-7 and Rauk, A., Why is the amyloid beta peptide of Alzheimer's disease neurotoxic? Dalton Trans., 2008(10): 1273-82. The discovery of the outward orientation of the lysine 28 residue is consistent with the high immunogenicity of this cyclic peptide comprising residues 25-29 (CGSNKGC), the side of lysine being solvent exposed, large and charged via an ε-amino group. The discovery of the outward orientation of the lysine 28 residue is consistent with authentic Aβ oligomers also displaying a similar lysine side-chain orientation in solvent in an antibody-accessible fashion. The serine 26, asparagine 27 and lysine 28 residues, SNK, located in the knuckle region of Aβ oligomers are all charged or polar, and have greater immunogenicity than small non-polar amino acids. The cyclic conformation of the SNK residues, located in the knuckle region of Aβ oligomers, form a novel conformational epitope that is solvent exposed and available for antibody binding. The discovery of this structurally constrained epitope at the surface of Aβ oligomers has advantageous properties for selective antibody binding.

In one aspect, the epitope is comprised of strongly polar/charged residues that are solvent-exposed and structurally constrained at the surface of Aβ oligomers. In another aspect, the structure of the novel conformation-specific epitope is dependent on a relatively-rigid spatial arrangement of the amino acid residues.

In one embodiment, the present conformational epitope having a constrained cyclic configuration is not present on the molecular surface of APP (amyloid precursor protein) thus limiting the autoimmune recognition of APP. The GSNKG motif of APP that is located at the cell surface of neurons and monocytes is largely unstructured. Conformation-specific antibodies binding to the novel conformational epitope having a constrained cyclic configuration have limited or no recognition of the unstructured GSNKG motif on cell surface APP. Antibodies recognizing the novel conformational epitope show little or no reaction with monomeric Aβ. In another aspect, antibodies recognizing the novel conformational epitope show little or no reaction with fibril Aβ.

In another embodiment, antibodies binding to the to the novel conformational epitope having a constrained cyclic configuration recognize the nonlinear epitope structure in between the subunits in the region of amino acids 25-29 of Aβ oligomers. The specificity of the antibodies to the novel conformational epitope enables the antibodies to specifically target the oligomeric form of Aβ and as such, avoid targeting monomeric Aβ and APP that are known to impact on neuronal and immune function and increase the availability of the antibody for binding as monomeric Aβ is present in much larger quantities than oligomeric Aβ. PCT/CA2011/000238.

The present invention provides for a peptide derived from Aβ which comprises a conformational epitope. This conformational epitope mimics the knuckle-like epitope in the misfolded, oligomeric Aβ. In one embodiment, the conformational epitope has an amino acid sequence comprising SNK. In another embodiment, the conformational epitope may be part of a cyclic peptide having an amino acid sequence comprising at least SNK. The peptide may be a cyclic peptide. The peptide may or may not be derived from Aβ. The conformational epitope may be part of a disulfide-linked cyclic peptide. The cyclization of the peptide may also through any other suitable covalent bonds. The present peptide may be cyclic, non-cyclic, branched, linear, or any other suitable form that can give a constrained configuration corresponding to the conformational epitope in oligomeric Aβ. This conformational epitope is described in detail in International Application No. PCT/CA2011/000238, the disclosure of which is incorporated by reference in its entirety.

As used herein, the term “conformational epitope” refers to an epitope where the amino acid residues take a particular three-dimensional structure. Antibodies which specifically bind a conformational epitope recognize the spatial arrangement of the amino acid residues of that conformational epitope.

The conformational epitope-containing peptide of the present invention may include a glycine residue located at either end of the SNK epitope sequence. The peptide may include glycine residues at both ends of the SNK epitope sequence. The glycine residue(s) may have limited or no contribution to the immunogenicity of the conformational epitope, or may relieve some steric tension inherent in the cyclization of the peptide. The peptide may include a glycine following lysine closer to the C terminus and a cysteine closer to the N terminus of the sequence. The conformational epitope may further include a cysteine followed by a native glycine on the N-terminal and a native glycine and a second glycine on the C terminal end. The conformation epitope may further include an N-terminal acetylated cysteine, followed by an additional cysteine and a native glycine and a cysteine at the C terminal end.

The present conformational epitope-containing peptides may comprise any standard (or natural) amino acids, non-standard amino acids, and/or amino acid analogues. Standard amino acids include the 20 L-amino acids identified in Table 1. Standard amino acids also include selenocysteine and pyrrolysine.

TABLE 1
Nomenclature and abbreviations of 20 standard L-amino acids

| Full Amino Acid name | Three-letter abbreviation | One-letter abbreviation |
|---|---|---|
| Alanine | Ala | A |
| Cysteine | Cys | C |
| Aspartic acid | Asp | D |
| Glutamic acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | T |

Non-standard amino acids may be naturally occurring or non-naturally occurring. Non-standard amino acids include any amino acid that may be incorporated into a polypeptide or result from modification of a natural amino acid. Several naturally occurring non-standard amino acids are known in the art, such as 4-hydroxyproline, 5-hydroxylysine, 3-methylhistidine, N-acetylserine, etc. In the present invention, an amino acid may be an L-amino acid or a D-amino acid. Amino acids in the present invention may be subject to any suitable modification, such as methylation, acetylation and/or phosphorylation.

Alteration may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Non-standard amino acids and amino acid analogues may be incorporated into a peptide during synthesis, or by modification or replacement of a natural amino acid after synthesis of a peptide.

An amino acid may be replaced by another amino acid on the basis of their structure and the general chemical characteristics of their R groups (side-chains). For example, an aliphatic amino acid can be replaced by another aliphatic amino acid; a hydroxyl or sulfur-containing amino acid can be replaced by another hydroxyl or sulfur-containing amino acid; a cyclic amino acid can be replaced by another cyclic amino acid; an aromatic amino acid can be replaced by another aromatic amino acid, a basic amino acid can be replaced by another basic amino acid; an acid amino acid can be replaced by another acid amino acid, etc. Alterations may comprise modifying an L-amino acid into, or replacing it with, a D-amino acid.

In one embodiment, the conformational epitope comprises an amino acid sequence of SNK in a cyclic constrained configuration or in a cyclic peptide. In one aspect, the conformational epitope comprises an amino acid sequence of at least SNK.

In another embodiment, the conformational epitope comprises an amino acid sequence of CSNKG (SEQ ID NO: 5) in a cyclic constrained configuration or in a cyclic peptide. In one aspect, the conformational epitope comprises an amino acid sequence of at least CSNKG.

In a third embodiment, the conformational epitope comprises an amino acid sequence of CGSNKGG (SEQ ID NO: 6) in a cyclic constrained configuration or in a cyclic peptide. In one aspect, the conformational epitope comprises an amino acid sequence of at least CGSNKGG.

In a fourth embodiment, the conformational epitope comprises an acetylated amino acid sequence of CCGSNKGC (SEQ ID NO: 7) in a cyclic constrained configuration or in a cyclic peptide. In one aspect, the conformational epitope comprises an amino acid sequence of at least CCGSNKGC.

In a fifth embodiment, the conformational epitope comprises an amino acid sequence of GSNK (SEQ ID NO: 1) in a cyclic constrained configuration or in a cyclic peptide. In one aspect, the conformational epitope comprises an amino acid sequence of at least GSNK.

In a sixth embodiment, the conformational epitope comprises an amino acid sequence of SNKG (SEQ ID NO: 2) in a cyclic constrained configuration or in a cyclic peptide. In one aspect, the conformational epitope comprises an amino acid sequence of at least SNKG.

In a seventh embodiment, the conformational epitope comprises an amino acid sequence of GSNKG (SEQ ID NO: 3) in a cyclic constrained configuration or in a cyclic peptide. In one aspect, the conformational epitope comprises an amino acid sequence of at least GSNKG.

In an eighth embodiment, the conformational epitope comprises an amino acid sequence of CGSNKGC (SEQ ID NO: 5) in a cyclic constrained configuration or in a cyclic peptide. In one aspect, the conformational epitope comprises an amino acid sequence of at least CGSNKGC.

TABLE 2
Sequences

| | Amino Acid |
|---|---|
| SEQ ID NO. | Sequence |
| 1 | GSNK |
| 2 | SNKG |
| 3 | GSNKG |
| 4 | CSNKG |
| 5 | CGSNKGC |
| 6 | CGSNKGG |
| 7 | CCGSNKGC |

The conformational epitope of the present antigenic peptide may comprise amino acid residues corresponding to residues 25 to 29 of oligomeric Aβ (1-40) or oligomeric Aβ (1-42).

The conformational epitope may comprise polar/charged amino acid residues that structurally constrained corresponding to the solvent-exposed amino acid residues located at the surface of Aβ oligomers.

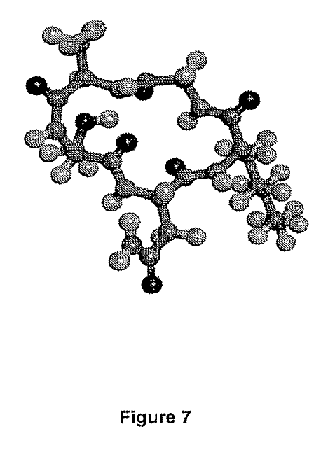

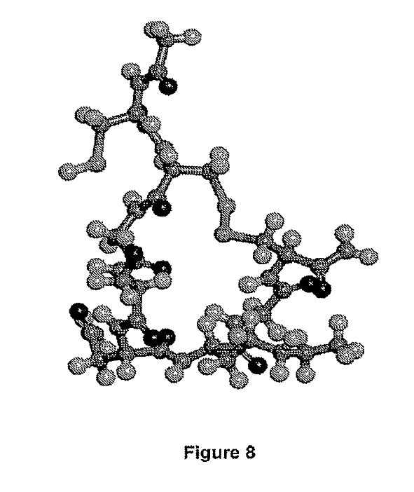

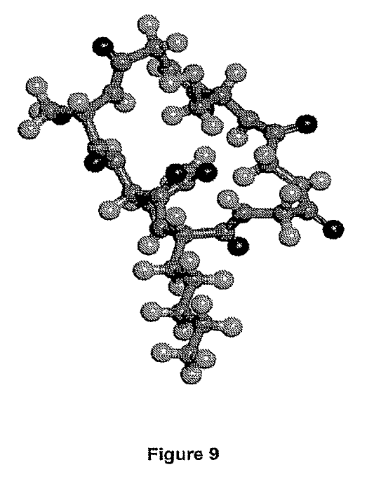

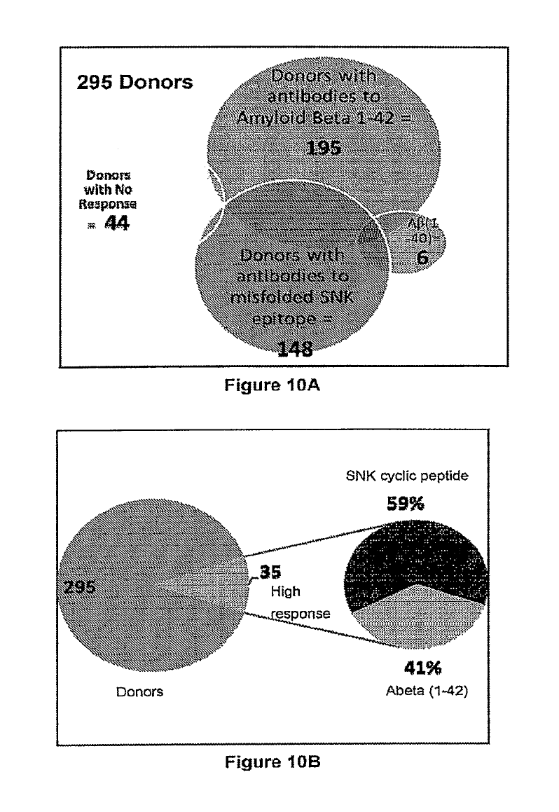

FIGS. 7, 8 and 9 illustrate three dimensional models of the CSNKG cyclic peptide, CGSNKGG cyclic peptide, and acetylated-CCGSNKGC cyclic peptide.

As used herein, the term “hyperimmune”, “hyperimmune preparation” or “hyperimmune composition” refers to a composition enriched with antibodies specific to one or more particular epitopes. The hyperimmune preparation can be isolated from normal or disease affected individuals such as those with dementing diseases, e.g., Alzheimer's disease. The present invention provides for a hyperimmune preparation enriched with antibodies specific to a conformational epitope of oligomeric Aβ. For example, the hyperimmune preparation is enriched with antibodies specific to a conformational epitope having an amino acid sequence comprising SNK (i.e., Ser-Asn-Lys) which may correspond to the cyclic peptide. The hyperimmune preparation may be enriched with antibodies specific to a conformational epitope having an amino acid sequence comprising at least SNK. The hyperimmune preparation binds with greater affinity to an oligomeric form of Aβ than to a non-oligomeric form of Aβ. The present hyperimmune preparations may contain an enriched population of antibodies specific to one or more of the conformational epitopes disclosed herein.

The present hyperimmune preparations may contain a high titer or concentration of antibodies specific to one or more of the conformational epitopes disclosed herein.

A hyperimmune preparation of the present disclosure comprises antibodies that may be derived from human or animal plasma after undergoing a series of processing steps. The first step comprises the screening of each donor's plasma to identify and collect plasma that demonstrates high titers or elevated serum levels of specific polyclonal antibodies, particularly high titers of antibodies specific to the present conformational epitope. Plasma donor samples having high antibody titers is pooled and fractionated. The primary component of the fractionated pooled plasma is IgG.

Various methods of preparing the hyperimmune preparation are discussed below.

The hyperimmune preparation may be prepared from animal plasma or serum, such as human plasma or serum. The animal may be non-immunized, naturally immunized or artificially immunized with the present conformational epitope. For example, plasma is collected from healthy donors. Plasma may be collected from the same species of animal as the subject to which the immunoglobulin preparation will be administered.

Plasma or serum may be obtained from animals immunized with a peptide containing a conformational epitope having an amino acid sequence comprising SNK via intramuscular, subcutaneous, intraperitoneal, or intraocular injection, with or without an adjuvant. As used herein, animals can include both human, non-human primates as well as other animals, such as, horse, sheep, goat, mouse, rabbit, dog, etc. Optionally, booster immunizations may be done and samples of serum are collected and tested for reactivity to the antigen in standard assays (described below). Once the titer of the animal has reached a plateau in terms of antigen reactivity, larger quantities of the antisera may be obtained readily either by periodic bleeding or exsanguinating the non-human animal.

For human plasma or serum, prospective donor's plasma is screened to determine the concentration of antibodies that are capable of binding to the conformational epitope. The prospective donor plasma may also be screened to determine the concentration of antibodies to Aβ peptides, oligomeric Aβ, and/or combinations thereof.

Pooled plasma is prepared by combining the donor plasma having a high antibody titer. The antibody titers in the pooled plasma are above a certain minimum level. For example, antibody titers to the conformational epitope may be in the range of about 1 to about 10000 mean fluorescence intensity (MFI), about 15 to about 10000 MFI, about 1 to about 2000 MFI, about 1 to about 1,000 MFI, about 5 to about 800 MFI, about 10 to about 600 MFI, about 1 to about 50 MFI, about 50 to about 100 MFI, about 50 to about 200 MFI, about 100 to about 200 MFI, about 200 to about 400 MFI, about 400 to about 600 MFI, about 600 to about 800 MFI, about 800 to about 1,000 MFI, or any range there between.

The use of MFI represents only one possible measure of antibody titer which may be determined using enzyme-linked immunosorbent assay (ELISA) or any other suitable method of measuring antibody titer (described below).

The pooled plasma is then fractionated to obtain the antibodies such as gamma globulin (IgG) fraction (including IgG1, IgG2, IgG3 and IgG4), IgM, IgA, IgD and/or IgE. Various methods of fractionating plasma well known in the art may be used in fractionating pooled plasma, including chromatography. For example, immunoglobulin can be purified by using Protein G, Protein A, Protein A/G, or Protein L. In one embodiment, immunoglobulin is purified by using Protein G, Protein A, Protein A/G, or Protein L columns or beads.

In one embodiment, the human plasma samples with the highest MFI as measured against the cSNK peptide conjugated to BSA by the MagPlex® experiments are selected for IgG purification. Plasma sample is first converted to serum. IgG is then purified from the serum through a High Trap™ Protein G column using an AKTA™ Purifier. IgG is eluted from the column at low pH and stored in PBS. The IgG is further buffer exchanged in 10 mM NaPO₄ (Sodium Phosphate, pH 6.2) and sterile filtered (0.22 μm).

Alternatively, IgG may be purified from plasma using the ethanol precipitation techniques, specifically variations of the Cohn process (Cohn et al., J. Am. Chem. Soc. 68:459-75 (1946)). This process fractionates plasma proteins based on their different solubility in varying ethanol concentrations, pH and temperature. Chromatography may also be used, including for example, ion-exchange chromatography or gel filtration chromatography. WO1999040939.

Non-limiting exemplary methods to purify an immunoglobulin fraction from plasma include: (a) the Cohn cold ethanol fractionation method or modifications thereto (Cohn et al., J. Am. Chem. Soc. 68:459-75 (1946); Huchet, J. et al, Rev. Fr. Transfus. 13:231, 1970; Chown, B. et al., Can. Med. Assoc. J. 100:1021, 1969; Jouvenceaux, A. et al., Rev. Fr. Transfus. 12 (suppl.): 341, 1969; Barandun, S. et al., Vox Sang. 7: 157-174, 1962); (b) ion-exchange chromatography (e.g. using DEAE-Sephadex) and modifications thereto (Cunningham, C. J. et al, Biochem. Soc. Trans. 8: 178, 1980; Hoppe, H. H. et al., Vox. Sang. 25: 308, 1973; Hoppe, H. H. et al, Munch. Med. Wochenschr, 109: 1749, 1967; Baumstark, J. S. et al., Arch. Biochem. 108:514, 1964; Canadian Patent number 1,168,152; Canadian Patent number 1,201,063); or (c) anion-exchange chromatography and modifications thereto (Canadian Patent No. 1,201,063). Non-limiting examples of other methods include: acid treatment (see Jouvenceaux, A. et al., Rev. Fr. Transfus. 12 (suppl.): 341, 1969), ultracentrifugation, or treatment with pepsin, plasmin, a sulfitolytic agent or beta-propriolactone (sec U.S. Pat. No. 4,160,763; Barandun, S. et al., Monogr. Allergy 9: 39-60, 1975; Stephan, Vox Sang. 28: 422-437, 1975; Wells, J. L. V. et al., Austr. Ann. Med. 18: 271, 1969; Baumgarten, W. et al., Vox Sang. 13: 84, 1967; Merler, E. et al., Vox Sang. 13: 102, 1967; Sgouris, J. T. et al., Vox Sang. 13: 71, 1967; Barandun, S. et al., Vox Sang. 7: 157-174, 1962; Nisonoff, A. et al, Science 132: 1770-1771, 1960). The immunoglobulins can be isolated from the blood by other suitable procedures, such as, ultracentrifugation, electrophoretic preparation, affinity chromatography, immunoaffinity chromatography, polyethylene glycol fractionation, etc. Oneley et al., J. Am. Chem. Soc. 71:541-50 (1949); Barundern et al., Vox Sang. 7:157-74 (1962); Koblet et al., Vox Sang. 13:93-102 (1967); U.S. Pat. Nos. 5,122,373 and 5,177,194.

Preparative steps can be used to enrich a particular isotype or subtype of immunoglobulin. For example, Protein A, Protein G or protein H sepharose chromatography can be used to enrich a mixture of immunoglobulins for IgG, or for specific IgG subtypes. (See generally Harlow and Lane, Using Antibodies, Cold Spring Harbor Laboratory Press (1999); Harlow and Lane, Antibodies, A Laboratory Manual, Cold Spring Harbor Laboratory Press (1988); U.S. Pat. No. 5,180,810.)

In one embodiment, immunoglobulin is prepared from gamma globulin-containing products produced by the alcohol fractionation and/or ion exchange and affinity chromatography methods well known to those skilled in the art. Purified Cohn Fraction II is commonly used. The starting Cohn Fraction II paste is typically about 95 percent IgG and is comprised of the four IgG subtypes. The Fraction II is further purified before formulation into an administrable product. For example, the Fraction II paste can be dissolved in a cold purified aqueous alcohol solution and impurities removed via precipitation and filtration. Following the final filtration, the immunoglobulin suspension can be dialyzed or diafiltered (e.g., using ultrafiltration membranes having a nominal molecular weight limit of less than or equal to 100,000 Daltons) to remove the alcohol. The solution can be concentrated or diluted to obtain the desired protein concentration and can be further purified by techniques well known to those skilled in the art.

In another embodiment, the present hyperimmune preparation is prepared by contacting plasma with one or more chromatographic separation columns to produce a purified IgG-rich fraction. Specifically, first plasma collected from animal or human is modified to the ionic strength and pH of the initial buffer used with the chromatographic separation column. Modified plasma is then applied to an anion exchange column which may contain an agarose cross-linked anionic exchange resin such as DEAE-Sepharose CL6B or DEAE-Biogel, and an IgG-rich fraction is obtained by eluting with an equilibrating buffer. The IgG-rich fraction may be concentrated, for example, by ultrafiltration. The concentrated IgG-rich fraction is then applied to a second different anion exchange column such as DEAE-Biogel or DEAE-Sephadex A-50. A purified IgG-rich fraction is isolated by elution with an appropriate equilibrating buffer which may be further purified using ultrafiltration.

Ultracentrifugation of the immunoglobulin-containing fraction, or treatment of immunoglobulin with pepsin, plasmin, a sulfitolytic agent or beta-propriolactone, may reduce the anti-complementary activity of the final preparation (see U.S. Pat. No. 4,160,763; Barandun, S. et al., Monogr. Allergy 9: 39-60, 1975; Stephan, Vox Sang. 28: 422-437, 1975; Wells, J. L. V. et al., Austr. Ann. Med. 18: 271, 1969; Baumgarten, W. et al., Vox Sang. 13: 84, 1967; Merler, E. et al., Vox Sang. 13: 102, 1967; Sgouris, J. T. et al., Vox Sang. 13: 71, 1967; Barandun, S. et al., Vox Sang. 7: 157-174, 1962; Nisonoff, A. et al., Science 132: 1770-1771, 1960). (see Canadian Patent number 1,168,152; Canadian Patent number 1,201,063; Cunningham, C. J. et al., Biochem. Soc. Trans. 8: 178, 1980; Hoppe, H. H. et al., Vox. Sang. 25: 308, 1973; Hoppe, H. H. et al., Munch. Med. Wochenschr. 109: 1749, 1967; Baumstark, J. S. et al., Arch. Biochem. 108:514, 1964).

The present hyperimmune preparation may contain greater than about 50%, greater than about 60%, greater than about 70%, greater than about 80%, greater than about 85%, greater than about 90%, greater than about 95%, or greater than about 99% IgG. The present hyperimmune preparation may contain greater than about 50%, greater than about 60%, greater than about 70%, greater than about 80%, greater than about 85%, greater than about 90%, greater than about 95%, or greater than about 99% IgM.

In one embodiment, the hyperimmune preparation is intravenous immunoglobulin (IVIG). IVIGs are purified IgG products. IVIG contains the pooled immunoglobulin G (IgG) from the plasma of a large number (e.g., pool sizes range from 1,000-60,000) of blood donors. U.S. Pat. No. 7,968,293. U.S. Patent Publication Nos. 2002/0114802; 2003/0099635 and 2002/0098182.

IVIG may be prepared using the methods disclosed herein. IVIG products may also be obtained from a number of commercial suppliers, including Baxter BioScience, Bayer Biological, Novartis, Talecris Biotherapeutics, Grifols USA, Octapharma USA, and ZLB Behring. Such sources include but are not limited to: Gammagard®. (Baxter Healthcare); BayRho-D®, products (Bayer Biological); Gamimune N® (Bayer Biological); Sandoglobulin I.V® (Novartis); Polygam S/D® (American Red Cross); Venoglobulin-S® (Alpha Therapeutic); Venoglobulin-S® (Alpha Therapeutic); and VZIG® (American Red Cross).

For the hyperimmune preparation, to enrich the antibodies specific to the present conformational epitope, the plasma, serum or IgG fraction may be affinity purified with the present peptides containing the conformational epitope. For example, the peptides are covalently coupled to a solid support such as agarose and used as an affinity ligand in the column to enrich or purify the specific antibodies.

Typically, during the purification process, plasma is filtered to remove pathogens; pathogens may also be inactivated. For example, the purified IgG may be treated with a solvent and detergent to inactivate lipid envelope viruses. Suitable solvents and detergents which may be used include Triton X-100 and tri(n-butyl) phosphate (Horowitz, B., Curr. Stud. Hematol. Blood Transfus. 56: 83-96, 1989). After the process, the solvents and detergents may be removed using conventional methods such as reverse phase chromatography.

Antibody titer or concentration may be measured by methods commonly known in the art. For example, Antibody titer may be measured by enzyme-linked immunosorbent assay (ELISA). In one embodiment, in an ELISA, antigens (e.g., conformational peptide-containing peptides) are first immobilized on a solid support (e.g., a plate). The primary antibody is added, which binds specifically to the test antigen coating the well. This primary antibody can be in a scrum sample, a plasma sample, a hyperimmune preparation or an IVIG sample, etc. A secondary antibody is added, which will bind the primary antibody. This secondary antibody often has an enzyme attached to it. A substrate for this enzyme is then added. Often, this substrate changes color upon reaction with the enzyme. The higher the concentration of the primary antibody present in the serum, the stronger the color change. Often, a spectrometer is used to give quantitative values for color strength. In quantitative ELISA, the optical density (OD) of the sample is compared to a standard curve, which is typically a serial dilution of a known-concentration solution of the target molecule. For example, if a test sample returns an OD of 1.0, the point on the standard curve that gave OD=1.0 must be of the same analyte concentration as the sample. Lequin R (2005), Enzyme immunoassay (EIA)/enzyme-linked immunosorbent assay (ELISA), Clin. Chem. 51 (12): 2415-8. Engvall E, Perlman P (1971), Enzyme-linked immunosorbent assay (ELISA), Quantitative assay of immunoglobulin G″. Immunochemistry, 8 (9): 871-4. Antibody titer may be determined by serial dilutions. A plasma sample containing antibody can be diluted serially. Using an appropriate detection method each dilution is tested for the presence of detectable levels of antibody. The assigned titer value is indicative of the last dilution in which the antibody was detected.

Antibodies titer may also be measured by in vitro multiplex bead-based immunoassays. Multiplex bead-based immunoassays, such as the Luminex® xMAP® technology, allow the measurement of one analyte or simultaneous measurement of multiple analytes using a library of antigen-containing (or epitope-containing) peptides (or proteins) coupled to color-coded beads. Each bead is identified by the unique wavelength it emits when excited by a laser. Quantitation is accomplished by a sandwich assay using a fluorescently labeled detection antibody with affinity to the specific analyte captured by the bead-coupled antibody beads. Excitation by a second laser reads the quantity of bound detection antibody. Houser, Brett, Using Bead-Based Multiplexing Immunoassays to Explore Cellular Response to Drugs, Drug Discover and Development, May 9, 2011. The beads that can be used in the Luminex® xMAP® immunoassays include MagPlex®, MicroPlex®, LumAvidin®, SeroMAP™ microspheres, etc. MagPlex® microspheres are superparamagnetic microspheres which are internally labeled with fluorescent dyes and contain surface carboxyl groups for covalent attachment of ligands (or biomolecules). Baker et al., Conversion of a Capture ELISA to a Luminex® xMAP® Assay using a Multiplex Antibody Screening Method, J. Vis. Exp., (65), e4084 10.3791/4084, DOI: 10.3791/4084 (2012). Fulton et al., Advanced multiplexed analysis with the FlowMetrix system. Clinical Chemistry, 43, 1749-1756 (1997). Carson et al., Simultaneous quantitation of 15 cytokines using a multiplexed flow cytometric assay, J. Immunol. Methods, 227, 41-52 (1999).

In one embodiment, to detect the presence of antibodies to the conformational epitope of a cyclic peptide in a sample, the cyclic peptides, having an amino acid sequence of at least SNK corresponding to a solvent-exposed, antibody accessible knuckle region of oligomeric Aβ, are coupled to MagPlex® microspheres based on the multiplexing xMAP® platform and analyzed on the MagPix® instrument (Luminex Corporation, Austin, Tex.). The sample is then contacted with cyclic peptide-coupled microspheres, an immunoassay is used to detect and quantify antibodies specific to the cyclic peptide. See Examples 3 and 4 below for details.

Different peptides, e.g., Aβ (1-42), Aβ (1-40), and cyclic peptides, may be coupled to different sets or regions of MagPlex® microspheres. Because each of these regions has a unique internal fluorescent dye, the immunoassay is able to discriminate between the antibodies specific to the different peptides.

Antibody titers can be measured by Biacore™ assay which measures protein-protein interaction and binding affinity based on surface plasmon resonance (SPR). Karlsson et al., Analysis of active antibody concentration, Journal of Immunological Methods, (1993) 166, 75-84. Markey F., Measuring concentration, Biajournal, 1999, 2: 8-11. Antibody titers can also be measured by radioimmunoassay.

In one aspect, the antibodies to the conformational epitope may be polyclonal. The hyperimmune preparation of the present disclosure comprises polyclonal antibodies that may target more than one epitope. In one aspect, the hyperimmune preparation of the present disclosure comprises antibodies to at least the conformational epitope. The targeting of multiple epitopes by the hyperimmune preparation is evident even when the pooled plasma is collected from individuals having elevated serum levels of specific polyclonal antibodies. The polyclonal antibodies of the hyperimmune preparation may target a range of different epitopes.

The present antibodies may be antibodies and/or fragments thereof. Antibody fragments include Fab, F(ab′)₂, scFv, disulfide linked Fv, Fc, or variants and/or mixtures thereof. The antibodies may be polyclonal, monoclonal, chimeric, humanized, single chain, or bi-specific. All antibody isotypes are encompassed by the present invention, including IgA, IgD, IgE, IgG, and IgM. Suitable IgG subtypes include IgG1, IgG2, IgG3 and IgG4.

The present disclosure provides a pharmaceutical composition comprising a pharmaceutically effective amount (e.g., a therapeutically effective amount) of the hyperimmune preparation described above and a pharmaceutically acceptable carrier. The pharmaceutical composition may be used for preventing the onset or reducing the severity or duration of Alzheimer's disease. As discussed above, the hyperimmune preparation comprises antibodies specific to a conformational epitope having an amino acid sequence comprising SNK.

The present compositions may be administered via various routes, including, but not limited to, intravenous, intramuscular, subcutaneous, oral, enteral, intranasal, intrapulmonary or inhalational.

The hyperimmune preparation of the present disclosure may be incorporated into a pharmaceutical composition suitable for, for example, parenteral administration. Preferably, the hyperimmune preparation will be prepared as an injectable solution containing an effective amount of the hyperimmune preparation. The injectable solution can be composed of either a liquid or lyophilized dosage form in a flint or amber vial, ampule or pre-filled syringe. Any suitable buffer may be used in the preparation of the pharmaceutical compositions. Examples of such buffers include but are not limited to, sodium succinate, sodium citrate, sodium phosphate or potassium phosphate. Cryoprotectants and bulking agents may be included for a lyophilized dosage form. Stabilizers may be used in both liquid and lyophilized dosage forms.

The pharmaceutical compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

The hyperimmune preparation produced by the processes above may be formulated with a wetting agent (non-ionic surface active agents) such as polysorbate 80, also known as TWEEN 80®. Non-ionic surface agents such as sorbitan esters or polyoxyethylene sorbitan esters of fatty acids (TWEEN® or SPAN® type surface active agents) may be added. Other stabilizers such as sodium chloride, mannitol, and/or L-glycine or L-histidine can also be used. The pH of the fraction may be adjusted. The resulting preparation may be sterilized, for example, by filtration.

Carbohydrates and their derivatives, such as glucose, sucrose, maltose, mannitol, sorbitol, etc., may be included in immununoglobulin formulations to adjust the tonicity of the preparation. Likewise, amino acids such as glycine or histidine may be added to improve storage stability of the protein. The immununoglobulin preparation may contain one or more non-ionic surface active agents in a physiologically compatible buffered medium.

Examples of pharmaceutically acceptable adjuvants are those used conventionally with peptide-based drugs, such as diluents, excipients and the like. Reference may be made to “Remington's: The Science and Practice of Pharmacy”, 21st Ed., Lippincott Williams & Wilkins, 2005, for guidance on drug formulations generally.

The selection of adjuvant depends on the intended mode of administration of the composition. In one embodiment of the invention, the compounds are formulated for administration by infusion, or by injection either subcutaneously, intramuscularly or intravenously, and are accordingly utilized as aqueous solutions in sterile and pyrogen-free form and optionally buffered or made isotonic. Thus, the compounds may be administered in distilled water or in saline, phosphate-buffered saline or dextrose solution.

A preparation according to the present invention may be in the form of a liquid formulation or may be lyophilized to form a powder formulation. The liquid formulation may be administered directly. The lyophilized powder formulation may be reconstituted in a physiologically compatible medium before administration.

By way of example, intravenously injectable immununoglobulin preparations may contain an immununoglobulin distributed in a physiologically compatible medium.

Suitable medium for the present compositions may be sterile water for injection (WFI) with or without isotonic amounts of sodium chloride. For example, diluents include sterile WFI, sodium chloride solution (see Gahart, B. L. & Nazareno, A. R., Intravenous Medications: a handbook for nurses and allied health professionals, p. 516-521, Mosby, 1997).

The immunoglobulin concentration in the pharmaceutical composition may range from about 0.1% (w/w) to about 30% (w/w), from about 0.5% (w/w) to about 20% (w/w), from about 1% (w/w) to about 15% (w/w), from about 2% (w/w) to about 3% (w/w), or from about 5% (w/w) to about 10% (w/w).

In certain embodiments, a hyperimmune preparation of the present disclosure may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present.

Creams, lotions and ointments may be prepared for topical application using an appropriate base such as a triglyceride base. Such creams, lotions and ointments may also contain a surface active agent. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods. Therapeutic compositions typically must be sterile and stable under conditions of manufacture and storage.

The present compositions may be used for prophylaxis and/or treatment of Alzheimer's disease. A therapeutically effective amount of the present pharmaceutical composition can be administered to an individual diagnosed with Alzheimer's disease. A pharmaceutically effective amount of the present pharmaceutical composition can be administered to an individual at risk for developing Alzheimer's disease.

The present invention provides for a method of treating Alzheimer's Disease in a subject by administering to the subject a pharmaceutically effective amount of the pharmaceutical composition described herein. An effective amount to be administered to the subject can be determined by a physician with consideration of individual differences in age, weight, disease severity, dose and frequency of administration, and individual response to the therapy.

In certain embodiments, the present composition can be administered to a subject at a dose ranging from about 1 μg to 1 mg/kg body weight, about 10 μg to 800 μg/kg body weight, about 20 μg to 600 μg/kg body weight, about 30 μg to 500 μg/kg body weight, about 10 μg to 400 μg/kg body weight, about 20 μg to 400 μg/kg body weight, about 60 μg to 100 μg/kg body weight, about 10 μg to 200 μg per kg body weight, about 100 μg to 200 μg/kg body weight, about 50 μg/kg body weight, or about 100 μg/kg body weight.

The dose may also range from about 10 mg/kg of body weight to about 5 g/kg body weight, about 5 mg/kg of body weight to about 2 g/kg body weight, about 50 mg/kg of body weight to about 4 g/kg body weight, about 100 mg/kg of body weight to about 3 g/kg body weight, about 0.1 g/kg body weight to about 1 g/kg body weight, about 0.2 g/kg body weight to about 0.8 g/kg body weight, about 0.2 g/kg of body weight to about 4 g/kg body weight, about 10 mg/kg of body weight to about 50 mg/kg body weight, about 0.2 g/kg body weight, about 0.4 g/kg body weight, about 0.8 g/kg body weight, about 5 mg/kg body weight to about 500 mg/kg body weight, at least about 10 mg/kg body weight, at least about 15 mg/kg body weight, at least about 20 mg/kg body weight, at least about 25 mg/kg body weight, at least about 30 mg/kg body weight or at least 50 mg/kg body weight, up to about 100 mg/kg body weight, up to about 150 mg/kg body weight, up to about 200 mg/kg body weight, up to about 250 mg/kg body weight, up to about 300 mg/kg body weight, or up to about 400 mg/kg body weight. In other embodiments, the doses of the immunoglobulin can be greater or less.

Using a mass/volume unit, the present composition can be administered to a subject at a dose ranging from about 0.1 mg/ml to about 2000 mg/ml, or any amount therebetween, for example 0.1, 0.5, 1, 2, 5, 10, 15, 20, 25, 30, 35, 40, 10 50, 60, 70, 80, 90, 0.100, 120, 140, 160 180, 200, 250, 500, 750, 1000, 1500, 2000 mg/ml, or any amount therebetween; or from about 1 mg/ml to about 2000 mg/ml, or any amount therebetween, for example, 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, mg/ml or any amount therebetween; or from about 10 mg/ml to about 1000 mg/ml or any amount 15 therebetween, for example, 10.0, 15.0, 20.0, 25.0, 30.0, 35.0, 40.0, 50.0 60.0, 70.0, 80.0, 90.0, 100, 120, 140, 160 180, 200, 250, 500, 750, 1000 mg/ml, or any amount therebetween; or from about 30 mg/ml to about 1000 mg/ml or any amount therebetween, for example 30.0, 35.0, 40.0, 50.0 60.0, 70.0, 80.0, 90.0, 100, 120, 140, 160 180, 200, 250, 500, 750, 1000 mg/ml.

The frequency of administration may range from about once every day, about three times a week, about twice a week, about once a week, about three times a month, about twice a month, about once a month, about once every other month, about once every 6 months, about once a year, about once every 2 years, or about once every 5 years, etc. The composition may also be administered in one or more doses per day.

The duration of administration can vary: it may be about 1 month, about 3 months, about 6 months, about 1 year, about 18 months, about 2 years, about 5 years, or about 10 years. In one embodiment, the treatment may last the remainder of a subject's natural life.

Effectiveness of the treatment may be assessed during the entire course of administration after a certain time period, e.g., about every 3 months, about every 6 months, about every 9 years, about every year, etc. The administration schedule (dose and frequency) may be adjusted accordingly for any subsequent administrations. U.S. Pat. Nos. 8,066,993 and 7,968,293.

The present compositions may be administered via various routes, including, but not limited to, intravenous, intramuscular, subcutaneous, transdermal, oral, enteral, intranasal, intrapulmonary or inhalational. When administering the compositions by injection, the administration may be by continuous infusion or by single or multiple bolus injections.

The pharmaceutical compositions may contain the antibodies together with one or more other active agents. Alternatively, the present compositions may be administered consecutively, simultaneously or in combination with one or more other active agents. Non-limiting examples of the active agents that may be used in combination with the present compositions include an acetylcholinesterase inhibitor (such as tacrine, rivastigmine, galantamine or donepezil) or an NMDA receptor antagonist (such as memantine). The other active agents that can be used in combination with the present composition include those that are useful for treating Alzheimer's disease or related dementias. For example, the hyperimmune preparation of the present disclosure may be co-formulated and/or coadministered with one or more additional antibodies that bind other targets.

Pharmaceutical techniques may also be employed to control the duration of action of the compositions/preparations of the present invention. Control release preparations may be prepared through the use of polymers to complex, encapsulate, or absorb the antibodies. WO1999040939. In certain embodiments, the composition takes the form of, for example, implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Also encompassed by the present invention are methods for diagnosing and screening Alzheimer's disease in a subject.

The level of antibodies specific to oligomeric Aβ may be used to determine a subject's susceptibility to Alzheimer's disease. In particular, the level of antibodies specific to the present conformational epitope may be used to determine a subject's susceptibility to Alzheimer's disease. A positive indication that disease is either present or the patient is at risk for developing Alzheimer's disease will be found when there is a decrease over time in the antibody titer. For example, there may be an about 1%, about 2%, about 3%, about 4%, about 5%, about 5% to about 10%, about 10% to about 20%, about 20% to about 30%, about 40% to about 50%, about 50% to about 60%, about 60% to 70%, about 70% to about 80% or about 80% to about 90% decrease in the antibody titer. The percent decrease is measured as a ratio of the titer at time t1 over t2, where t1 and t2, may differ by about 1 month, about 2 months, about 3 months, about 4 months, about 5 months, about 6 months, about 6 months to about 12 months, about 8 months to about 16 months, about 16 months to about 24 months, or about 20 months to about 30 months. Other ranges are possible and can be determined by one or ordinary skill in the art. In this embodiment, t1 can represent a control sample. Control samples can also be derived from non-immune serum or from individuals who do not show the presence of any antibody reacting with the epitope.

The present disclosure provides for a method of diagnosing Alzheimer's Disease in a subject comprising the steps of: (a) obtaining a biological sample from the subject; (b) quantifying in the sample the level of antibodies specific to a conformational epitope having an amino acid sequence comprising at least SNK; and (c) comparing the level of the antibodies in step (b) with a control sample. A different level (e.g., a lower level) of the antibodies specific to the conformational epitope in the sample compared to the control sample are indicative of a diagnosis of Alzheimer's disease in the subject.

The present disclosure also provides for a method of diagnosing Alzheimer's Disease in a subject comprising the steps of: (a) obtaining a biological sample from the subject; (b) quantifying in the sample the level of antibodies specific to one or more of Aβ(1-40), Aβ(1-42), and a conformational epitope having an amino acid sequence comprising at least SNK; and (c) comparing the level of the antibodies in step (b) with a control. A different level (e.g., a lower level) of the antibodies specific to one or more of Aβ(1-40), Aβ(1-42), and the conformational epitope in the sample compared to the control are indicative of a diagnosis of Alzheimer's disease in the subject.

The present disclosure provides for a method of diagnosing Alzheimer's disease in a subject comprising the steps of: (a) obtaining a sample from the subject; and (b) detecting in the sample the level of antibodies specific to a conformational epitope having an amino acid sequence comprising at least SNK, wherein minimal or no antibodies detected in step (b) is indicative of a diagnosis of Alzheimer's disease in the subject.

In one embodiment, a subject demonstrating an absence of antibodies to the conformational epitope is at a higher risk of developing Alzheimer's disease.

The present disclosure provides for a method of predicting a subject's risk of developing Alzheimer's disease comprising the steps of (a) obtaining a biological sample from the subject; (b) quantifying in the sample the level of antibodies specific to a conformational epitope having an amino acid sequence comprising at least SNK; and (c) comparing the level of the antibodies in step (b) with a control sample. A different level (e.g., a lower level) of the antibodies specific to the conformational epitope in the sample compared to the control sample indicates that the subject is at risk of developing Alzheimer's disease.

The present disclosure also provides for a method of predicting a subject's risk of developing Alzheimer's disease comprising the steps of: (a) obtaining a biological sample from the subject; (b) quantifying in the sample the level of antibodies specific to one or more of Aβ(1-40), Aβ(1-42), and a conformational epitope having an amino acid sequence comprising at least SNK; and (c) comparing the level of the antibodies in step (b) with a control. A different level (e.g., a lower level) of the antibodies specific to one or more of Aβ(1-40), Aβ(1-42), and the conformational epitope in the sample compared to the control indicates that the subject is at risk of developing Alzheimer's disease.

Non-limiting examples of the control sample include a sample from one or more Alzheimer's disease-free subjects; a sample from one or more subjects having Alzheimer's disease; a stored dataset comprising results generated from studies of one or more Alzheimer's disease-free subjects; a stored dataset comprising results generated from studies of one or more subjects having Alzheimer's disease; a sample from the same subject obtained a time period ago where the time period may be about 6 months, about 1 year, about 2 years, about 3 years, about 4 years, about 5 years, about 6 years about 7 years, about 8 years, about 9 years, or about 10 years ago; a stored dataset comprising results generated from the same subject a time period of about 6 months, about 1 year, about 2 years, about 3 years, about 4 years, about 5 years, about 6 years about 7 years, about 8 years, about 9 years, or about 10 years; or a clinically selected control. Alternatively, the control sample may be a sample containing a minimum concentration of antibodies to the conformational epitope, or a maximum concentration of antibodies to the conformational epitope. The biological sample may be plasma, a tissue, cells, biofluid, or combinations thereof. The biofluid may be cerebrospinal fluid (CSF) or blood.

When applied in vitro, the detection method entails analysis of a biological sample of body fluid or tissue or organ sample from a subject. A tissue or organ sample, such as that obtained from a solid or semi-solid tissue or organ, may be digested, extracted or otherwise rendered to a liquid form. A biological sample or samples may be taken from a subject at any appropriate time, including before the subject is diagnosed with, or suspected of having Alzheimer's disease or a related dementia, during a therapeutic regimen for the treatment or amelioration of symptoms of that disease or disorder, after death of the subject (regardless of the cause, or suspected cause). Alternately, a biological sample may include donated body fluid or tissue, such as blood, plasma or platelets when in care of a centralized blood supply organization or institution. In addition, a biological sample may be taken from a healthy subject to act as a control.

The present methods may be used for the monitoring of Alzheimer's disease progression within a single patient, or monitoring the effectiveness of a therapeutic agent to slow, stabilize, or reverse the progression of Alzheimer's disease.

In one embodiment, the level of antibodies detected to the cyclic peptide in a normal healthy population may be compared to the level of antibodies detected to the cyclic peptide in a known population of subjects with Alzheimer's disease to provide an indication of disease progression or to provide an early diagnosis of Alzheimer's disease.

Also encompassed by the present invention is an article of manufacture comprising packaging material and a pharmaceutical composition. The composition comprises a pharmaceutically acceptable adjuvant and a therapeutically effective amount of the hyperimmune preparation described above. The packaging material may be labeled to indicate that the composition is useful to treat Alzheimer's disease. The packaging material may be any suitable material generally used to package pharmaceutical agents including, for example, glass, plastic, foil and cardboard.

In another embodiment, a kit for diagnosing Alzheimer's disease or related dementias is provided. The kit comprises a hyperimmune preparation as described above, along with instructions for use of the hyperimmune for the diagnosis or screening of Alzheimer's disease. The instructions may include, for example, dose concentrations, dose intervals, preferred administration methods, methods for immunological screening or testing, or the like. The hyperimmune may further be coupled to a detection reagent. Examples of detection reagents include secondary antibodies, such as an anti-human antibody, an anti-mouse antibody, an anti-rabbit antibody or the like. Such secondary antibodies may be coupled with an enzyme that, when provided with a suitable substrate, provides a detectable colorimetric or chemiluminescent reaction. The kit may further comprise reagents for performing the detection reaction, including enzymes such as proteinase K, blocking buffers, homogenization buffers, extraction buffers, dilution buffers or the like.

Plasma is collected from high titer donors. B cells are flow sorted based on binding to peptides which contains the present conformational epitope (e.g., comprising the sequence of at least SNK). Monoclonal antibodies are then cloned from the sorted B cells.

Human monoclonal antibodies can be obtained, for example, from human hybridomas (see, e.g., Cote et al., Proc. Natl. Acad. Sci. USA 80:2026-30 (1983)) or by transforming human B cells with EBV virus in vitro (see, e.g., Cote et al., supra).

Alzheimer patients receiving the present compositions can be in the early, middle or late stages of the disease progression, with mild, moderate or severe symptoms. In other cases, individuals suspected of beginning to develop Alzheimer's disease or considered at risk of developing this disease may also receive such treatment, so that their progression towards onset of the disease may be halted or reversed, or their risk of developing the disease may be diminished or eliminated. In other words, the anti-Alzheimer treatment can be applied as a method of preventing Alzheimer's disease or inhibiting or delaying the onset of the disease in at-risk individuals with no or only suspected symptoms. U.S. Pat. No. 8,066,993.

The present compositions and methods can be used in any animal subject, such as a mammal. A subject may further be a human. Specific animals include rats, mice, dogs, cats, cows, sheep, horses, rabbits, goats, pigs, birds or primate. A subject may further be an experimental animals or a transgenic animal.

### EXAMPLES

### Example 1

### Coupling of Cyclic Peptides to MagPlex®-C Microspheres

Various peptides derived from Aβ were coupled to MagPlex®-C microspheres, which are superparamagnetic carboxylated xMAP® microspheres. Aβ (1-42) was coupled to MagPlex®-C microsphere region 26, Aβ (1-40) was coupled to microsphere region 28, cyclic peptide cSNK (sequence: CGSNKGC) conjugated to BSA was coupled to microsphere region 18, and BSA was couple to microsphere region 30. Aβ (1-42) and Aβ (1-40) were purchased from Bachem (Torrance, Calif.). The cyclic peptide was provided by Dr. Neil Cashman's Laboratory, Brain Research Centre (Vancouver, BC).

The cyclic peptide comprised a conformational epitope having an amino acid sequence of at least SNK corresponding to a solvent exposed, antibody accessible knuckle region of oligomeric Aβ described in PCT Application PCT/CA2011/000238.

MagPlex®-C microspheres (regions 018, 026, 028 and 030) were obtained from Luminex Corporation (Austin, Tex.). The volume required for each type of MagPlex®-C microsphere was calculated according to a 1× scale coupling reaction requiring 1.25×10⁶ microspheres. The volume was adjusted accordingly when a higher scale coupling reaction was required. The calculated volume of stock microspheres were washed with water by centrifugation and re-suspended in 4-morpholineethanesulfonic acid (50 mM, MES buffer), pH 6.0. The microspheres were then activated by the addition of N-hydroxysulfosuccinimide (Sulfo-NHS) and 1-Ethyl-3-[3-dimethylaminopropyl] carbodiimide hydrochloride (EDC), both at a concentration of 50 mg/mL in water. The microspheres were incubated for 20 minutes at room temperature after which two washes were performed using MES (100 mM) buffer, pH 6.0. The microspheres were re-suspended in MES (100 mM) buffer, pII 6.0 and 10 μg for each 1× scale coupling reaction of the cyclic peptide coupled with BSA was added. The peptide (or protein) and the microspheres were allowed to couple either for two hours at room temperature or overnight at 2-8° C. in the dark. Following coupling, the microspheres were blocked for 30 min at room temperature with phosphate buffered saline (PBS) containing 1% bovine serum albumin (BSA) and 0.05% azide, pH 7.4. The microspheres were then washed twice with phosphate buffered saline with Tween 20 (PBS-T) by centrifugation. Once the microspheres were washed, they were re-suspended in PBS containing 1% BSA and 0.05% azide, pH 7.4 (storage buffer). Each coupled microsphere type was counted with the use of a hemocytometer and then stored at a temperature between 2−8° C. in the dark until ready for use. The coupling of the cyclic peptide to the microspheres was confirmed using a murine mAb.

### Example 2

### Collection of Plasma Samples

295 plasma samples from 295 donors were tested in this study. All donors were adults ranging from 18 to 75 in age residing in Canada. The donors consisted of 159 males and 136 females. The plasma samples were collected between Apr. 15, 2010 and Feb. 15, 2011.

For each immunoassay conducted, as described in Example 3 below, a total of 46 plasma samples were tested per assay. Each plasma sample was diluted 1 in 20 (in PBS-T) prior to analysis and tested in duplicate.

The average of the two intermediate values was reported as the result.

### Example 3

### MagPlex® Multiplex Immunoassays

Immunoassays were performed on the coupled microspheres described in Example 1 on the plasma samples collected.

Each assay was performed by diluting the peptide-coated microspheres in PBS-T such that when 100 μL was added to each well of a 96-well microplate, 3000 microspheres of each microsphere type was present per well. Following the addition of the microspheres to the microplate, 50 μL of each diluted plasma sample was added to the microspheres. An IVIG product was used as a control for each assay and 50 μL of the IVIG product was diluted (1 in 100 in PBS-T) and added to the remaining wells. The plasma sample and peptide-coated microsphere mixture was allowed to incubate at room temperature on a plate shaker for 60 minutes in the dark. Unbound antibodies and remaining matrix were removed by placing the microplate on the magnetic separator plate for 1 minute and then dumping the supernatant by inverting the microplate, attached to the magnetic separator plate, into the sink. The peptide-coupled microspheres with bound antibodies were washed twice with 100 μL per well of PBS-T for 1 minute per wash. A volume of 100 μL per well of the detection R-phycoerythrin AffiniPure goat anti-human IgG (PE-IgG) antibody was added at a diluted concentration of 2 μg/mL and allowed to incubate at room temperature on a plate shaker for 30 min in the dark. The excess detection antibody was removed using the magnetic separator plate again for 1 minute and dumping the supernatant as described above. The microspheres containing the bound material were washed twice with 100 μL per well of PBS-T for 1 minute per wash. The microspheres were then re-suspended with 100 μL of PBS-T and measured using the MAGPIX® analyzer instrument.

### Example 4

### MagPlex® Analysis of Cyclic Peptide Coupled Microsphere

The immunoassays described in Example 3 for the cyclic peptide coupled microspheres were analyzed to determine a Luminex mean fluorescence intensity (MFI) value.

Data from each assay was measured with the MAGPIX® analyzer and Luminex mean fluorescence intensity (MFI) values were processed with the xPONENT software (provided by Luminex Corporation). The BSA-coupled microspheres were used as a background reference against the cyclic peptide-coupled microsphere by subtracting their Luminex MFI values. The reference subtracted Luminex MFI values were used as the final reportable values for the cyclic peptide results.

Each type of peptide-coupled microsphere used was able to be measured all at once due to the different regions of internal fluorescent dyes within each microsphere type. The MAGPIX® is able to discriminate between the antibodies binding of the different regions of the various microspheres.

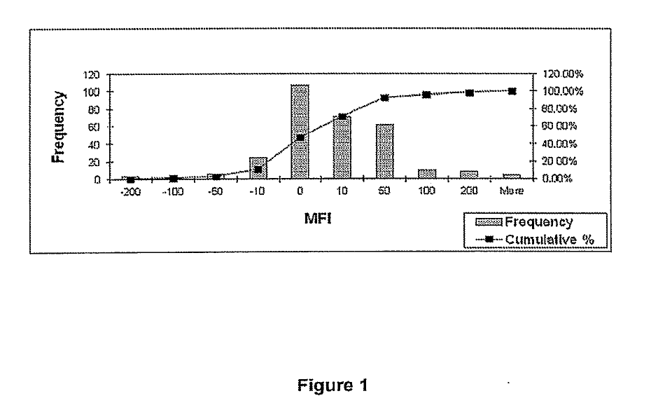

FIG. 1 shows the histogram distribution of the Luminex MFI results (BSA reference subtracted) for the cyclic peptide-coupled microspheres obtained for each plasma sample tested. The results indicate that from the plasma sample population tested, about 47% had negative Luminex MFI values due to the BSA reference binding having a stronger MFI signal than the cyclic peptide Luminex MFI signal. The remaining plasma samples, about 53%, were positive for the cyclic peptide.

Out of this positive population, 133 plasma samples or about 86% had an MFI value between 0 to 50; 18 plasma samples or about 12% had an MFI value between 50 to 200; and 4 plasma samples or about 3% had an MFI value above 200. Of the 4 plasma samples having the highest MFI values, 3 of the plasma samples were from males of age 50 to 65. The other plasma sample having a high MFI value was from a female of age 29.

### Example 5

### MagPlex™ Analysis Aβ (1-42), Aβ (1-40), Cyclic Peptide Coupled Microspheres

The immunoassays described in Example 3 for Aβ (1-42), Aβ (1-40), cyclic peptide coupled microspheres were analyzed to determine a Luminex mean fluorescence intensity (MFI) value.

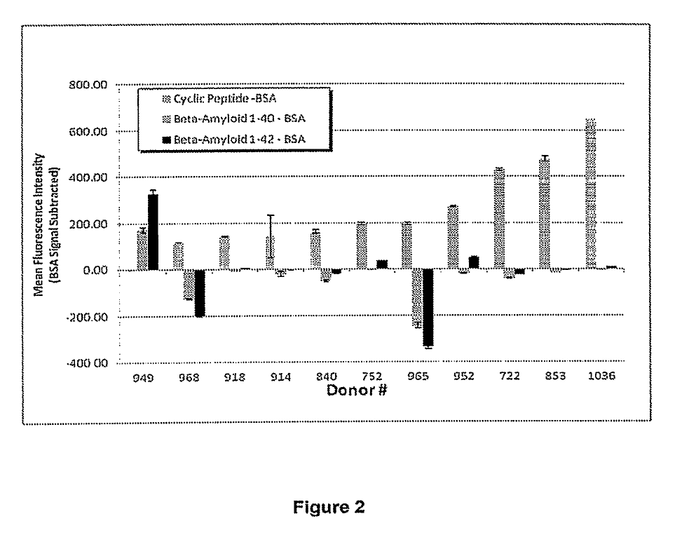

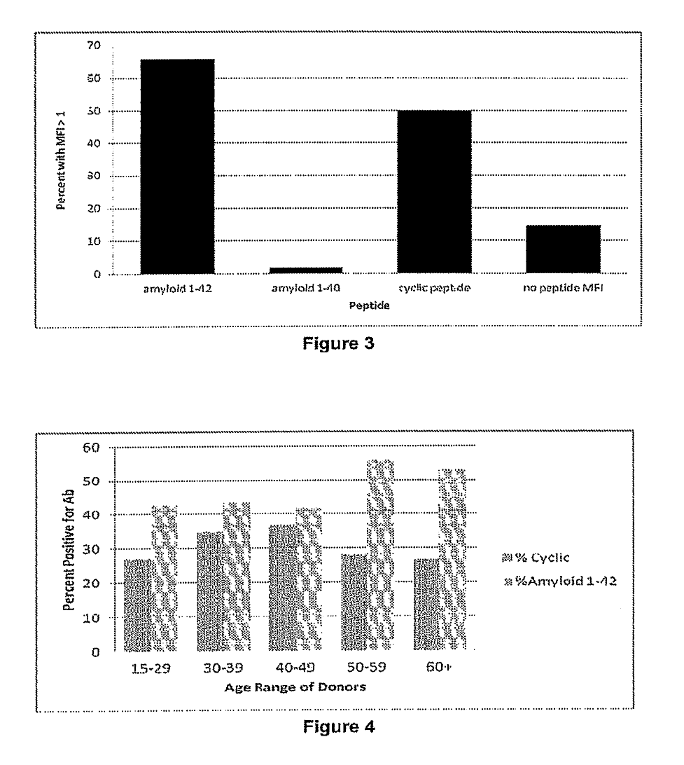

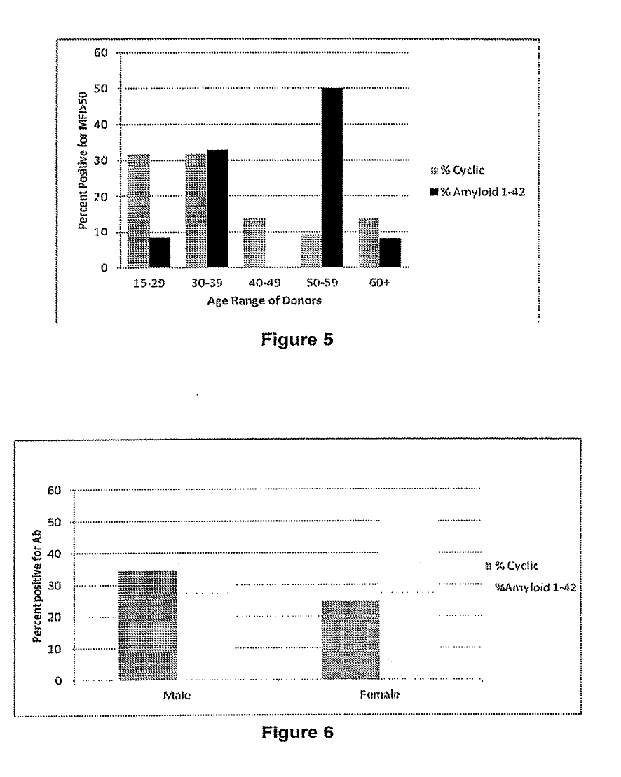

Approximately 300 samples from 300 donors were screened for antibodies binding Aβ (1-42), Aβ (1-40), and the cyclic peptide. FIGS. 2, 3, 4, 5 and 6 graphically illustrate the results which indicate that there is a subset of the screened donor population that have antibodies that recognize the cyclic peptide, that sometimes recognize the Aβ (1-42) peptide, and that less frequently recognize the Aβ (1-40) peptide. These results illustrate that despite amino acid homology existing across the Aβ (1-42), Aβ (1-40) peptides and the cyclic peptide, the antibody titers for specific donors are not consistent as would normally be expected. The results indicate that of the ten donor samples showing the highest antibody titer to the cyclic peptide, none of these samples showed positive a antibody titer to the Aβ (1-40) peptide and only of these samples showed a positive antibody titer for the Aβ (1-42) peptide despite the fact that the actual amino acid sequence of the cyclic peptide is fully present in both the 1-40 and 1-42 beta-amyloid peptides. This demonstrates that the three-dimensional epitope structure of the cyclic peptide binds a specific set of antibodies.

### Example 6

### Naturally Occurring Antibodies to Misfolded Amyloid-β Peptides in Normal Adults

### Methods

Using a customized MagPlex® immunoassay and ELISA, we screened 295 normal human plasma samples, ranging from 18 to 75 years of age for the presence of antibodies to Aβ peptides: Aβ (1-42), Aβ (1-40), and cyclic SNK peptide that mimics misfolded Aβ oligomers. Superparamagnetic Carboxylated xMAP® Microspheres (Luminex Corporation, Austin, Tex.) were coupled to the peptides, and analyzed on a MAGPIX analyzer system.

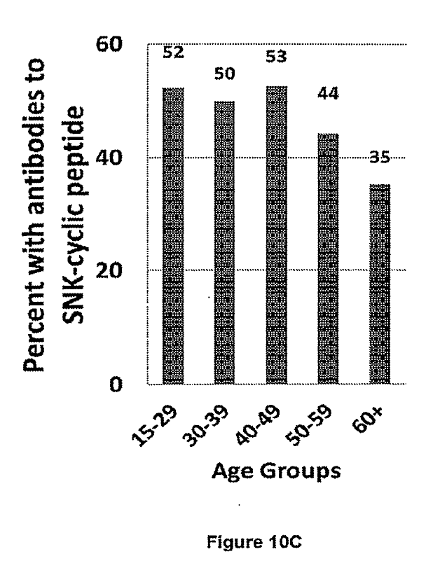

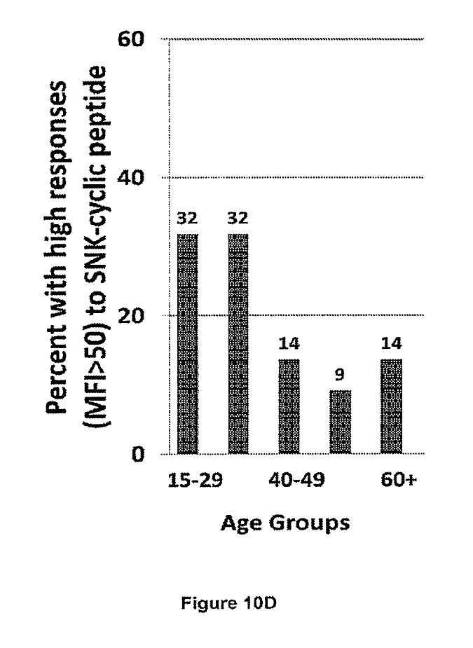

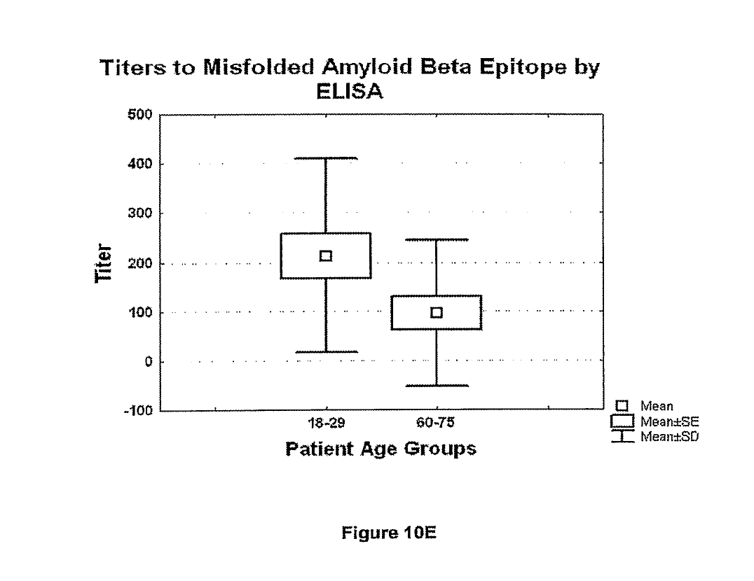

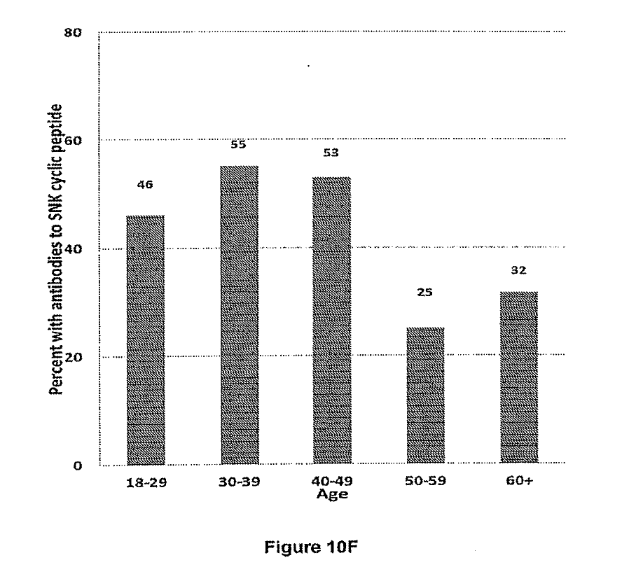

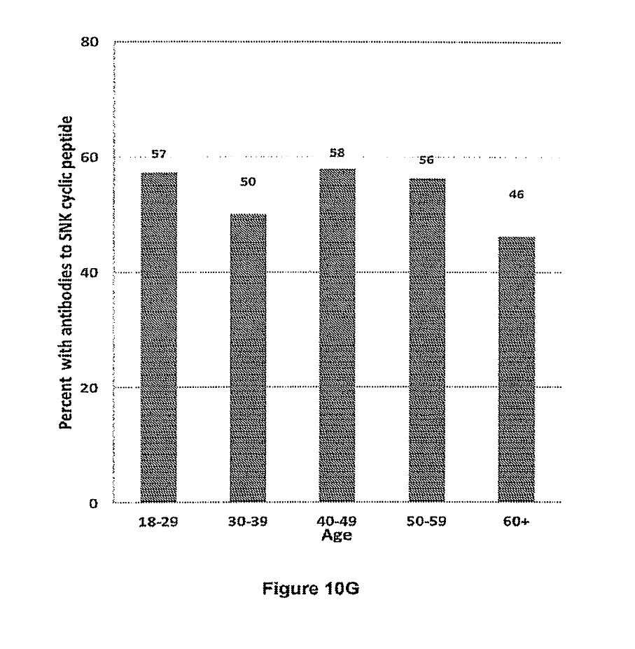

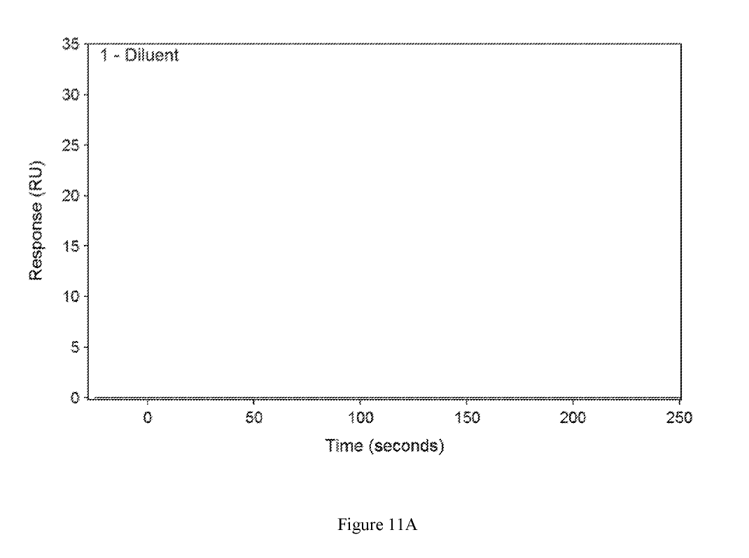

Normal adult population exhibited antibodies to Amyloid-β peptides: Aβ(1-42), Aβ(1-40), and cyclic SNK peptide (FIG. 10A). Out of 295 donors, antibodies to Aβ(1-42) were detected in 195 individuals; antibodies to Aβ(1-40) were detected in 6 individuals; antibodies to misfolded SNK epitope were detected in 148 individuals. 44 donors did not exhibit antibodies to any of the three Aβ peptides.

In 35 donors exhibiting high levels of Aβ reactivity, 59% of high responders have antibodies to misfolded SNK epitope, 41% of high responders have antibodies to Aβ(1-42) (FIG. 10B).

It was observed that younger population had higher levels of naturally occurring antibodies to misfolded amyloid-β epitope. Donors were separated into different age groups and the percentage with antibodies to the misfolded Aβ epitope SNK-cyclic peptide was assessed. The younger demographic population exhibited higher percentage of donors with antibodies to the misfolded amyloid 3 epitope (FIG. 10C). When high responding individuals were assessed, Somers' d test showed a reverse trend for age and levels of antibodies to SNK-cyclic peptide (FIG. 10D).

To quantitatively compare the antibody titers between the youngest and oldest donor age groups, ELISA and MagPlex immunoassay with SNK cyclic peptide were completed. Younger donors had higher levels of antibodies to the misfolded Aβ SNK-cyclic epitope (FIG. 10E).

The reverse trend for age and antibody levels is more profound in the female population (FIGS. 10F and 10G).

### CONCLUSION

The level of the antibodies specific to the conformational epitope in the human donors correlate inversely with age, particularly in females. These results demonstrate a differential immune response to the various Aβ epitopes in a normal population that may provide a marker of AD progression and an association with age. The screening study of AD patients will assess the distribution of antibodies in patients compared to a normal age-matched population.

### Example 7

### Biacore™ Assay Measuring Binding of Antibodies in Human Serum to cSNK Peptide and Abeta Oligomer

### Methods

The normal human plasma samples with the highest MFI as measured against the cSNK peptide conjugated to BSA by the MagPlex® experiments were selected for IgG purification. For comparison, normal human plasma samples with lower MFI to cSNK peptide were also purified for IgG. Each human plasma sample was converted to serum. IgG was purified from the scrum through a High Trap™ Protein G column using an AKTA™ Purifier. IgG was eluted from the column at low pH and stored in PBS. The IgG was further buffer exchanged in 10 mM NaPO₄ (Sodium Phosphate, pH 6.2) and sterile filtered (0.22 μm).

All Biacore™ experiments were performed using a Biacore™ 3000 instrument, Certified-grade CM5 sensor chips were activated with a mixture containing equimolar amounts of EDC (N-ethyl-N′-(dimethylaminopropyl) carbodiide and NHS (N-hydroxysuccinimide). Synthetic Abeta (1-42) oligomers and the conformationally-constrained Abeta oligomer epitope mimic, cSNK peptide that is conjugated to BSA, were appropriately diluted and covalently coupled to separate flow cells on the sensor chip surface. Residual unreacted sites on the sensor chip surface were then quenched. A reference surface to account for non-specific binding was accordingly generated by immobilizing BSA on an adjacent flow cell.

To verify the integrity, activity and specificity of the immobilized ligands, the mouse monoclonal antibody (mAb) 5E3 and an IgG1 isotype control were diluted and injected over the sensor chip surface. Normal human plasma samples that were IgG-enriched, were then tested for binding to the immobilized ligands, and bound antibodies further characterized by sequentially injecting mouse anti-sera to human IgG1, IgG2, IgG3, IgG4, IgG and IgM.

In a parallel set of experiments, a customized MagPlex® immunoassay and cSNK peptide coupled to Superparamagnetic Carboxylated xMAP® Microspheres were also employed for the detection of antibodies to Aβ peptides in these samples.

All MagPlex® experiments were performed using a MAGPIX® analyzer system. Superparamagnetic Carboxylated xMAP® microspheres were suspended in MES (2-(4-morpholino)ethanesulfonic acid) and activated with an equal mixture of sulfo-NHS (N-hydroxysulfosuccinimide) and EDC (1-Ethyl-3-[3-dimethylaminopropyl] carbodiimide hydrochloride). After washing with MES, conformationally-constrained Abeta oligomer epitope mimic, cSNK peptide conjugated to BSA, was appropriately diluted and covalently coupled to the microspheres. Following coupling, the microspheres were blocked with BSA/azide, washed and resuspended in BSA/azide for storage. Reference BSA microspheres to account for non-specific binding was accordingly generated.

For each MagPlex® experiment, diluted normal human plasma samples were added to the coupled microspheres and allowed to incubate. Unbound sample antibodies were removed and remaining microspheres were washed. PE-IgG (R-phycoerythin AffiniPure goat anti-human IgG) was added and allowed to incubate. Excess PE-IgG was removed, remaining microspheres were washed and resuspended for measuring using the MAGPIX analyzer instrument. The response was measured as a mean fluorescence intensity (MFI) values.

### Results

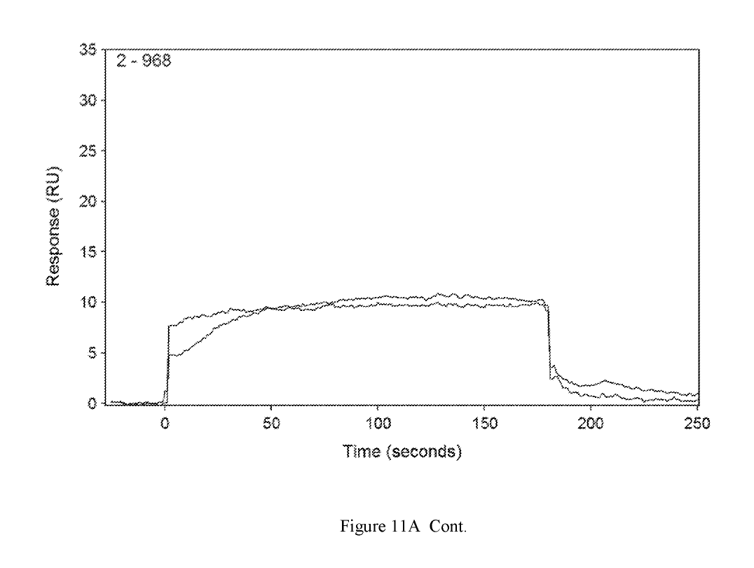

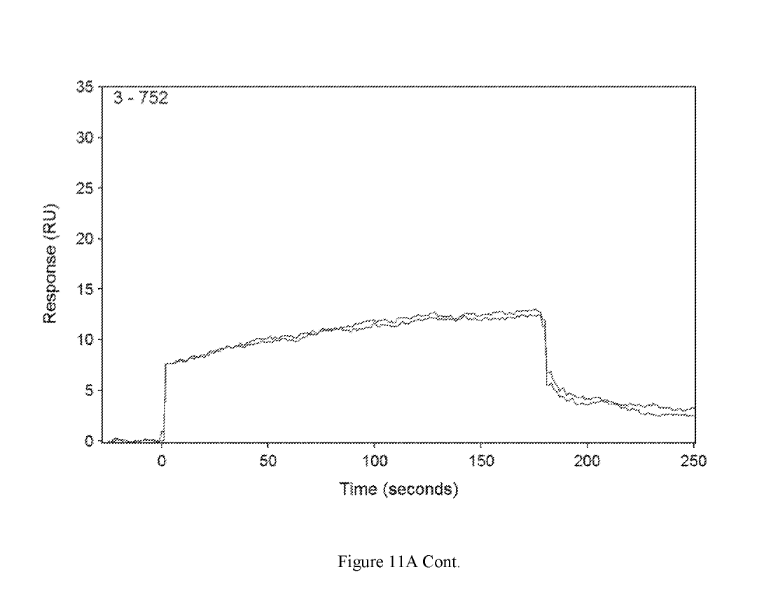

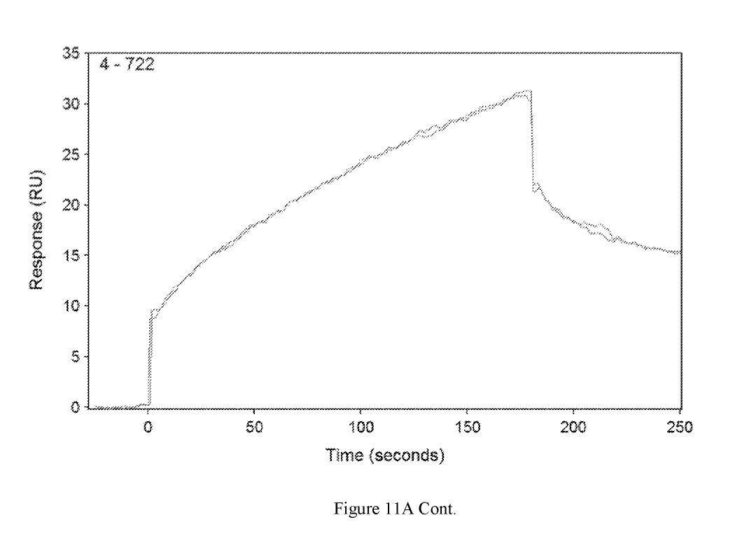

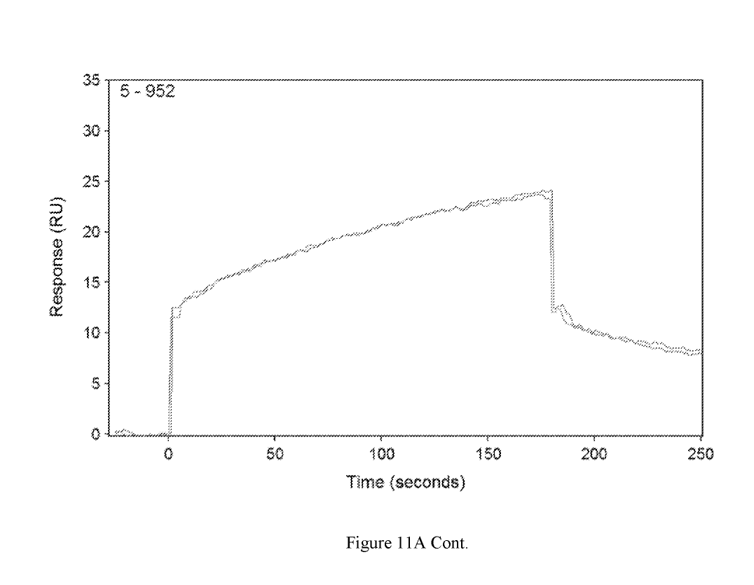

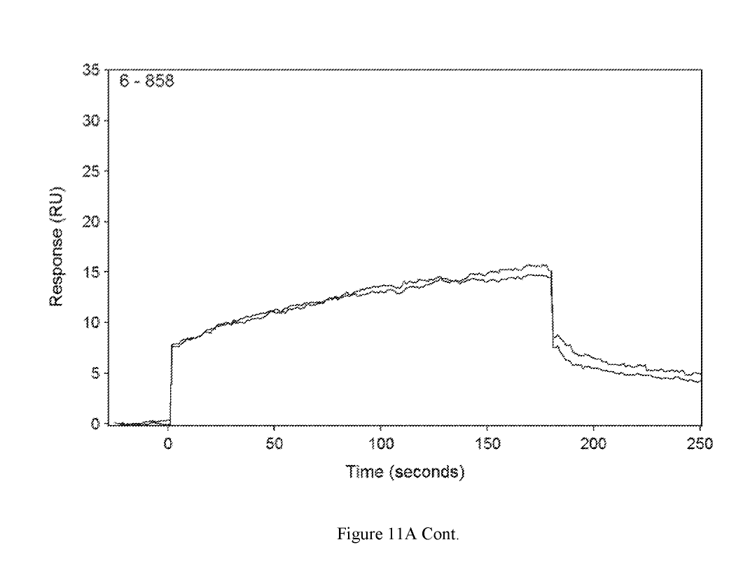

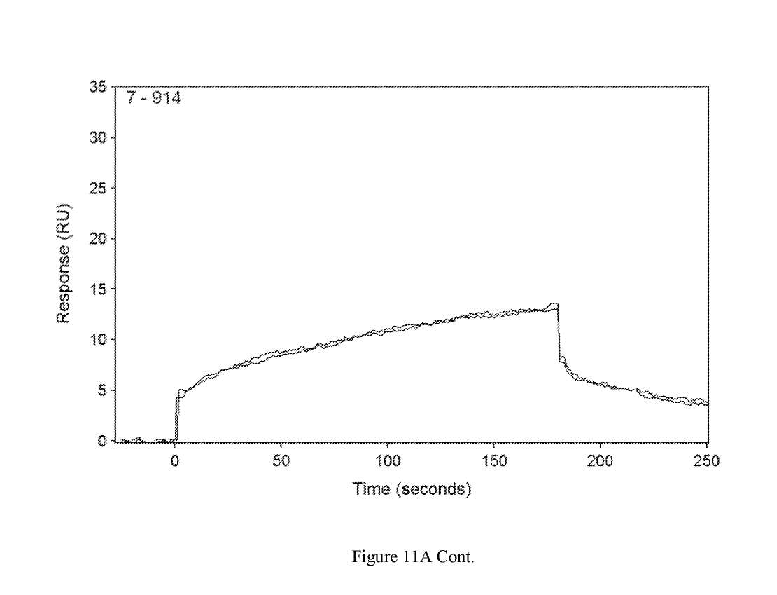

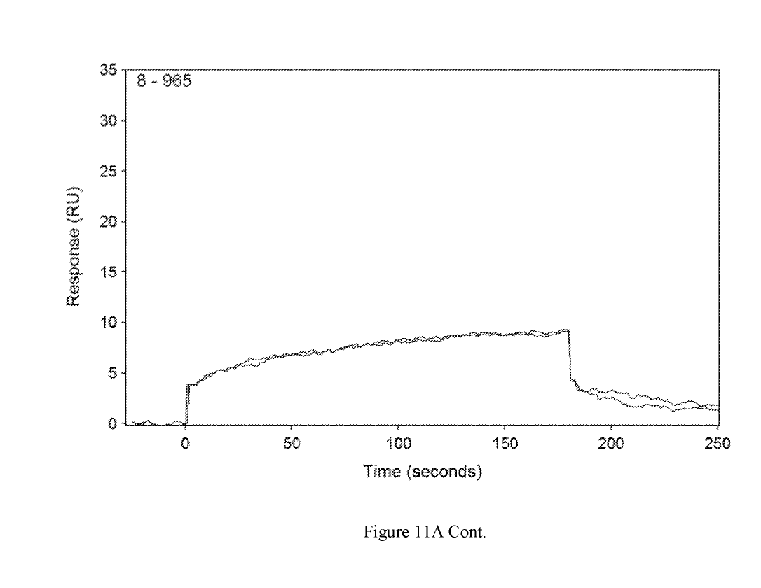

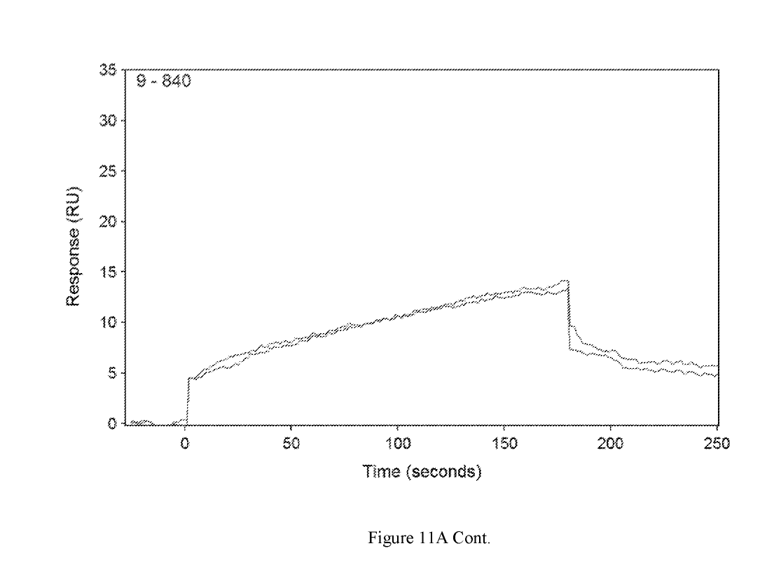

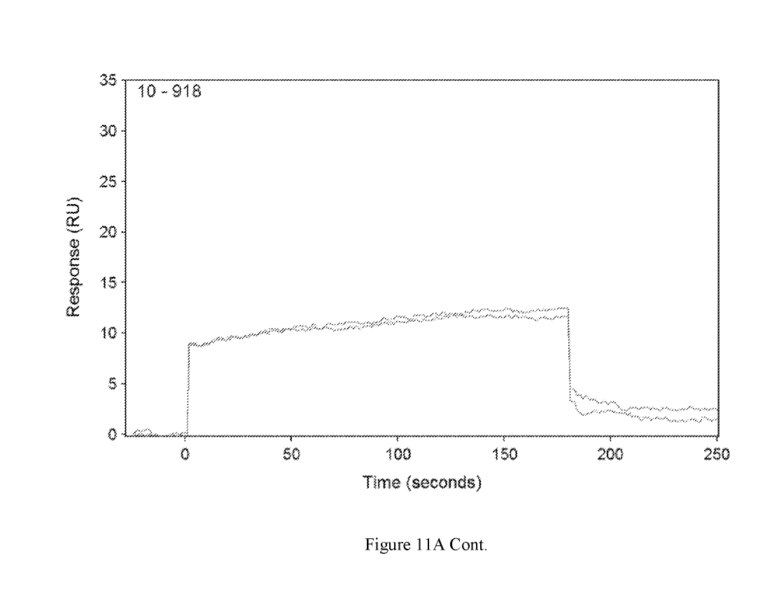

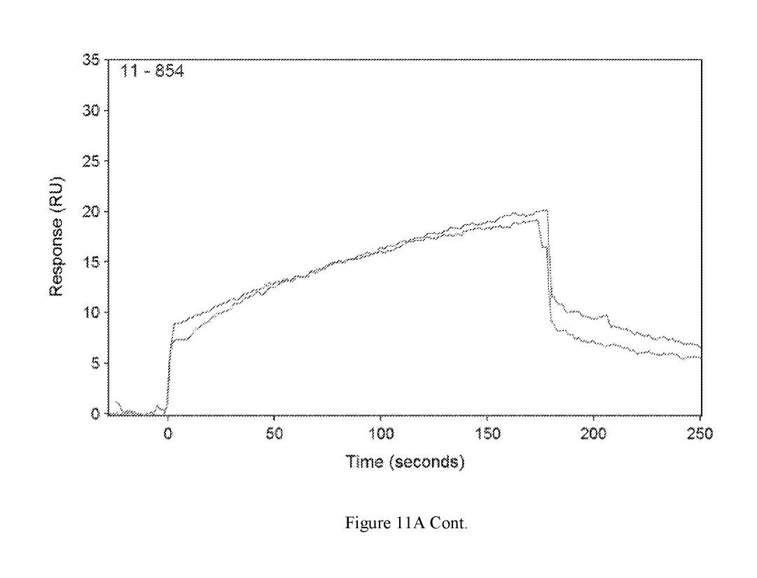

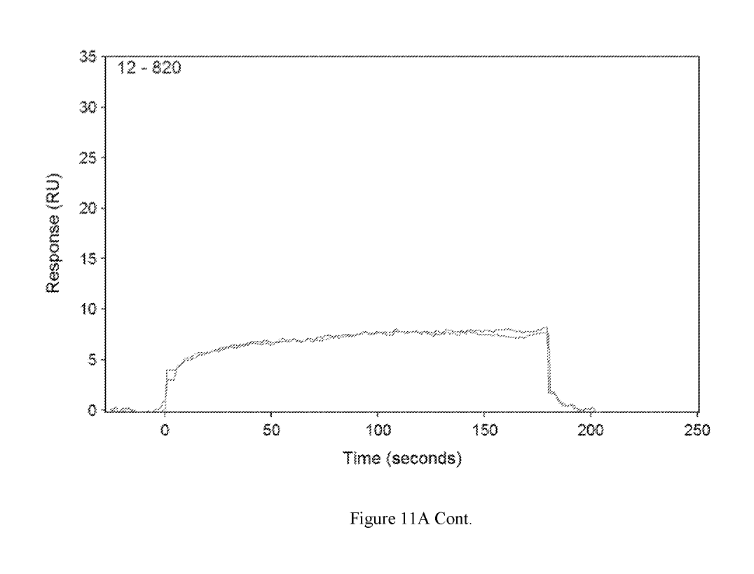

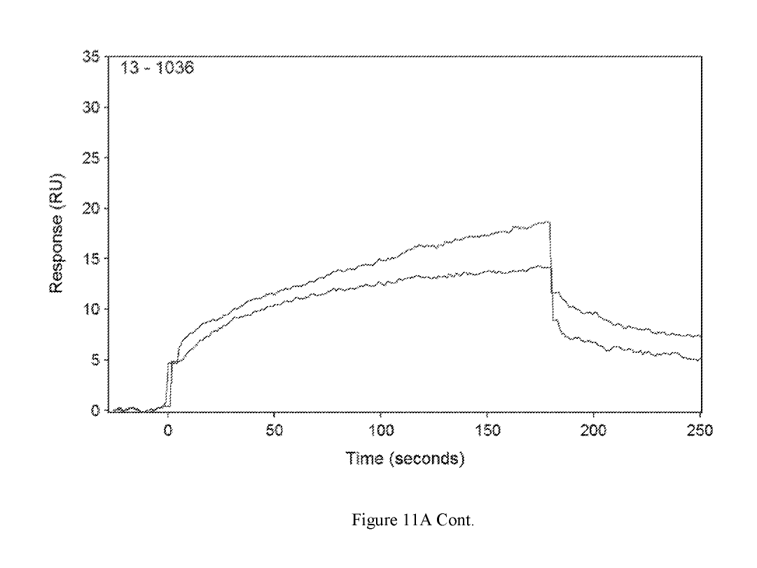

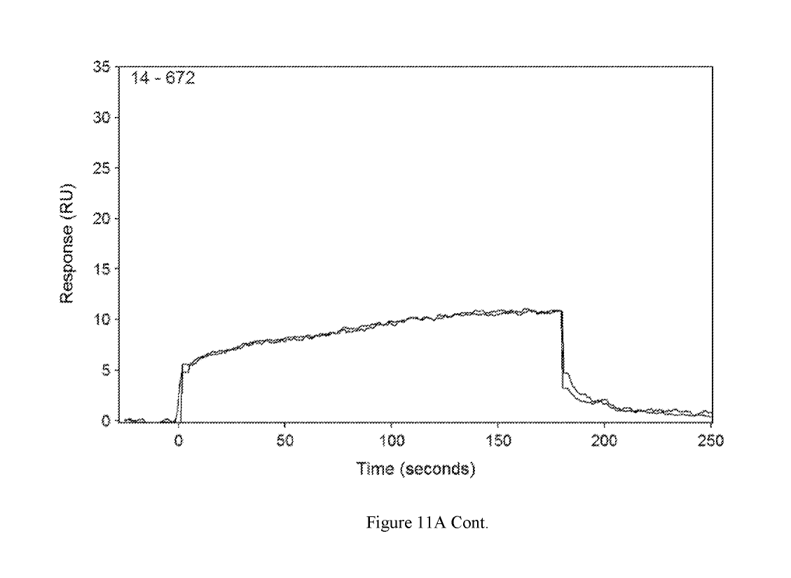

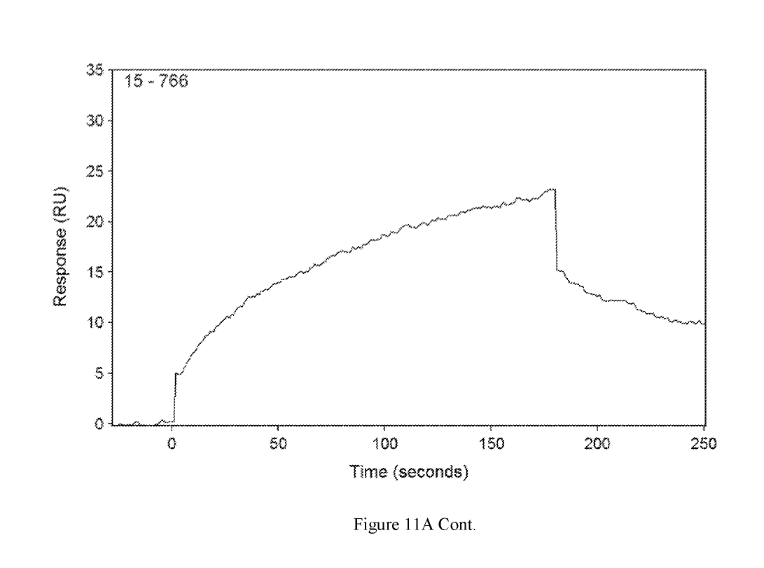

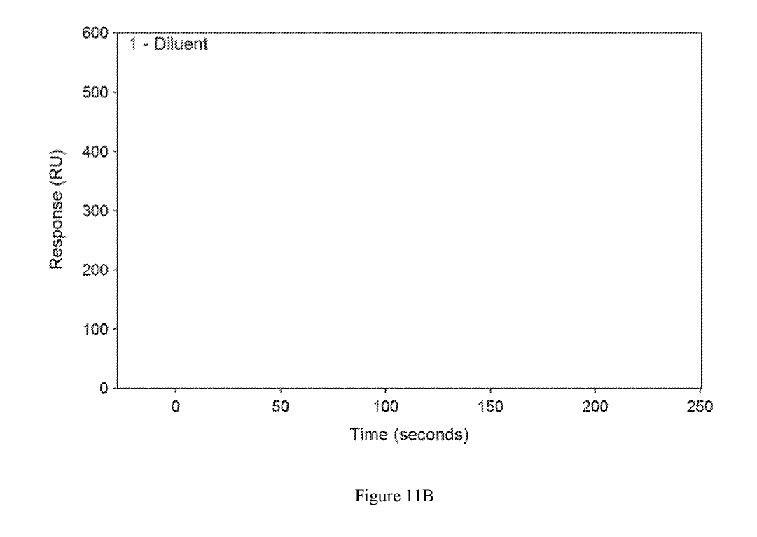

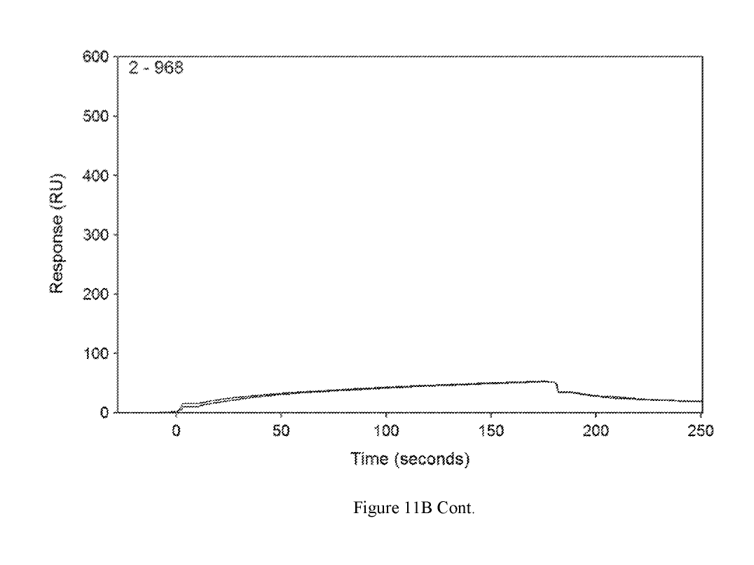

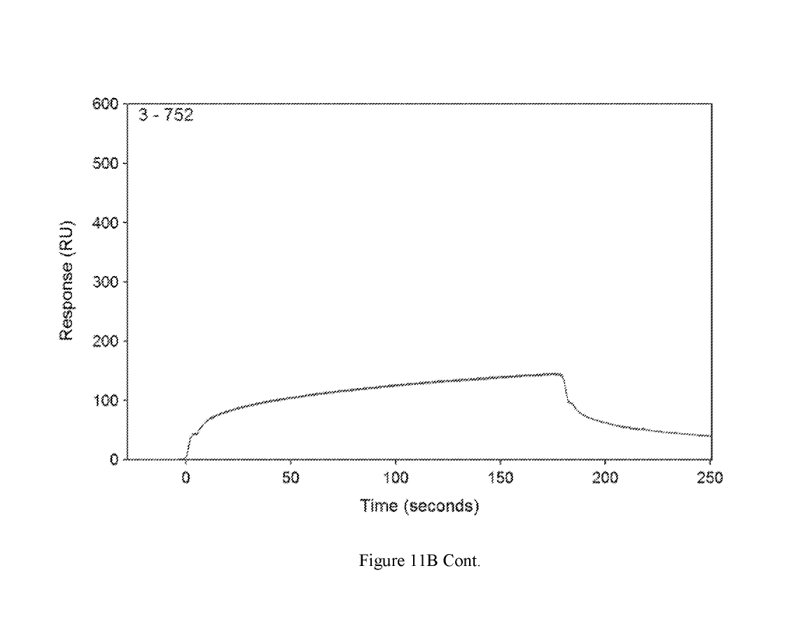

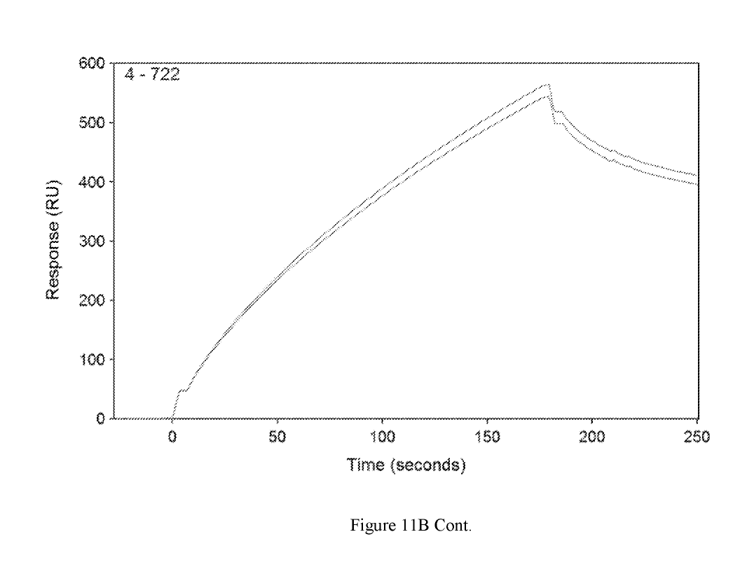

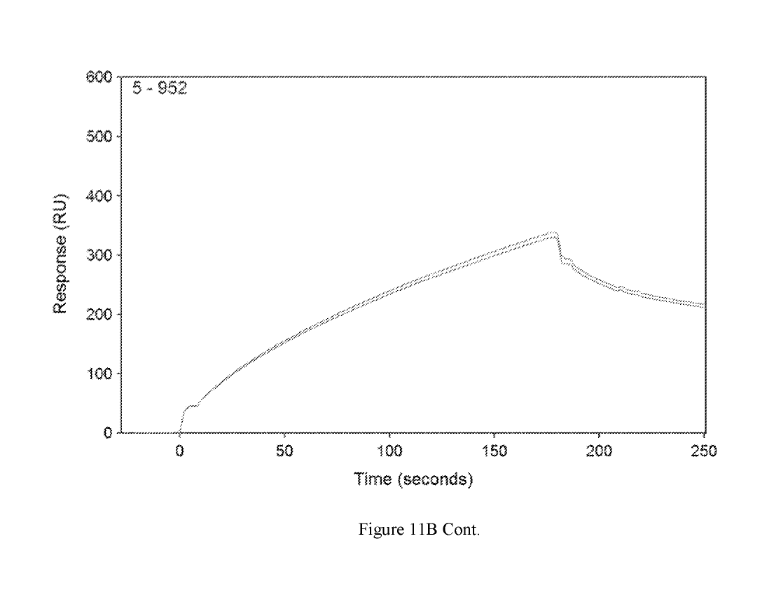

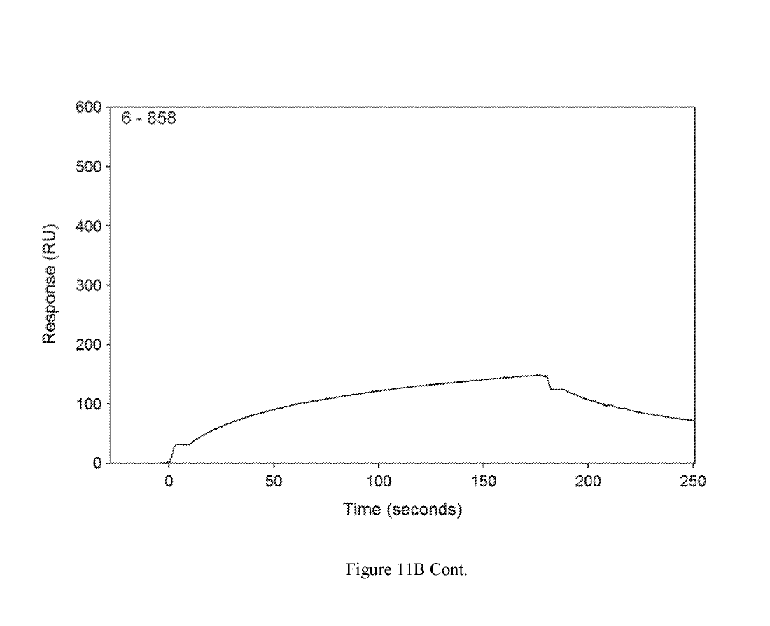

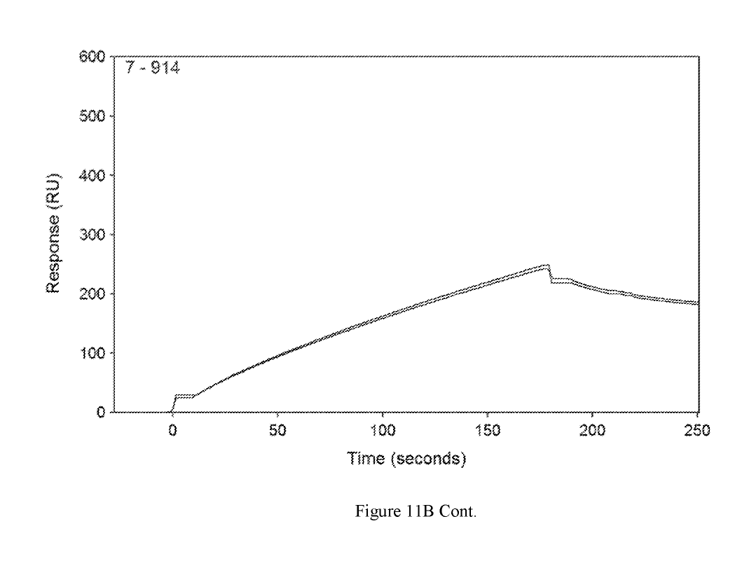

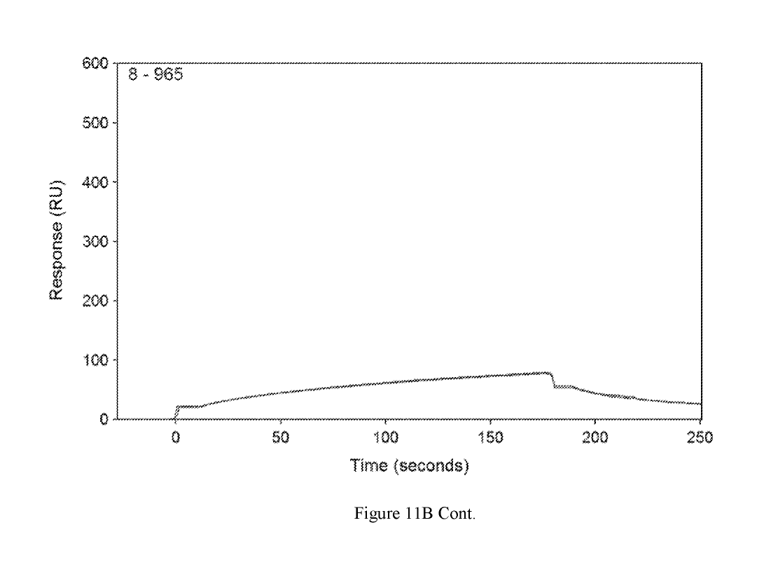

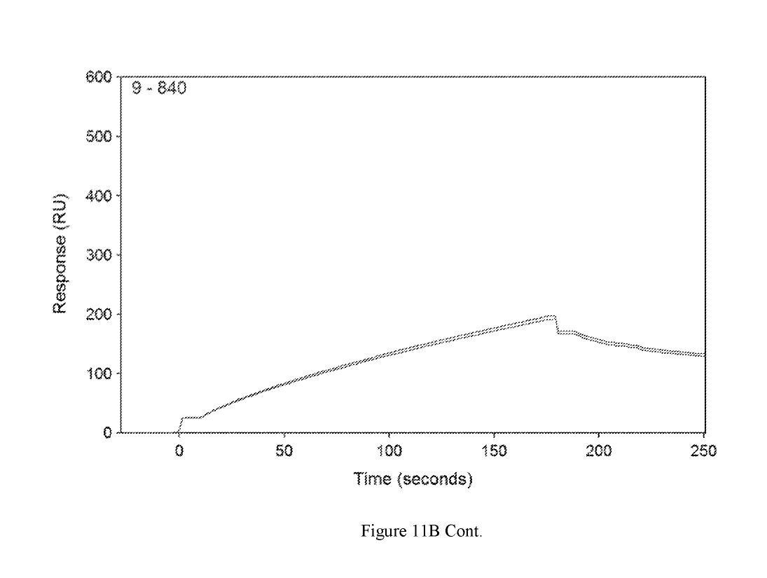

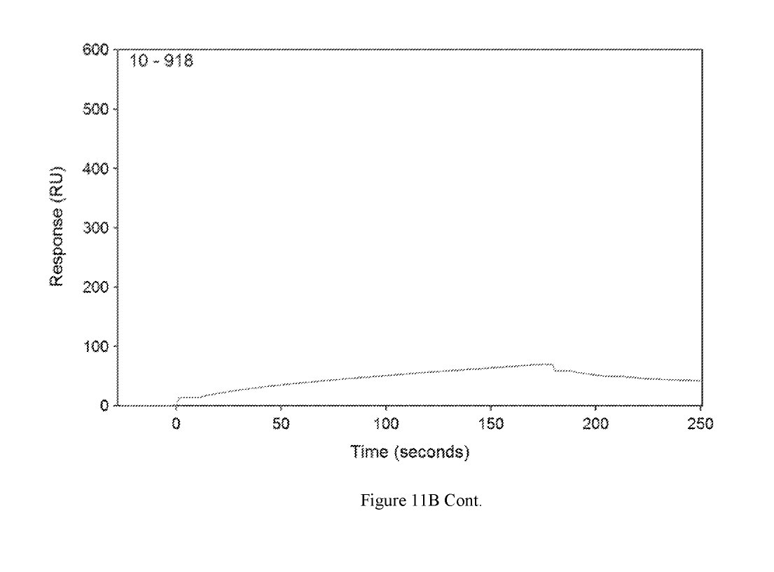

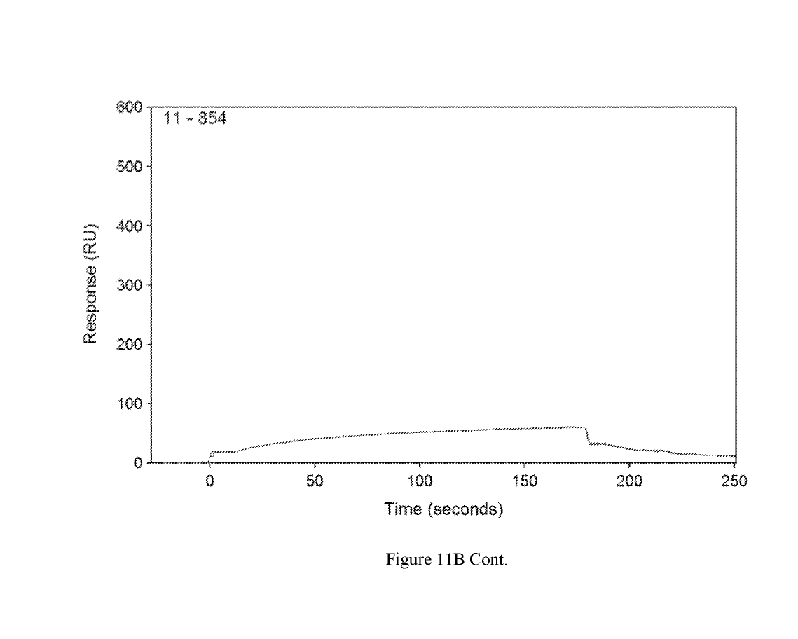

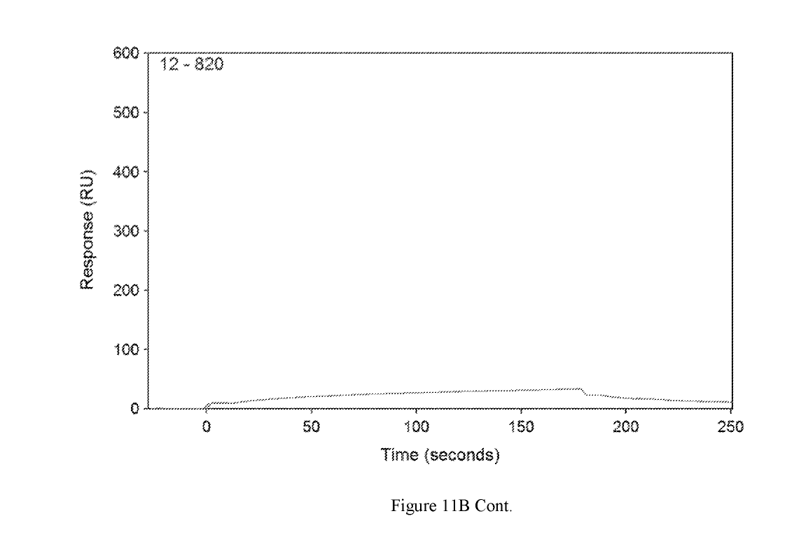

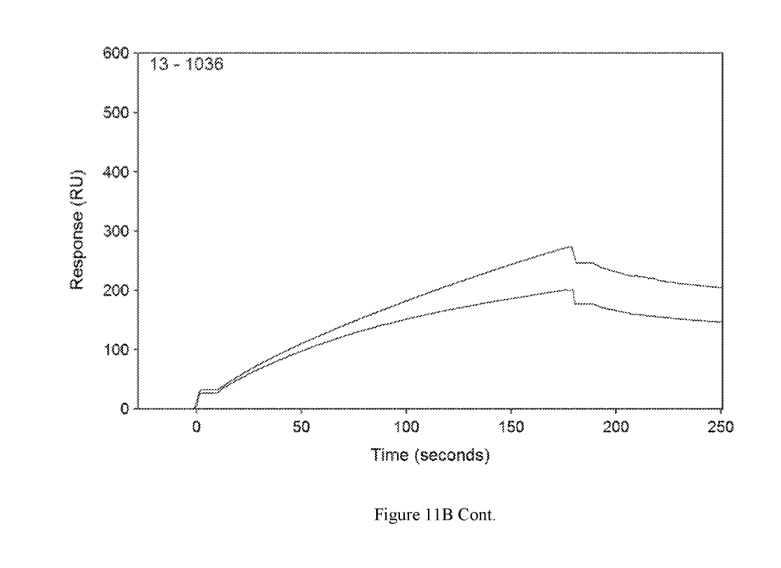

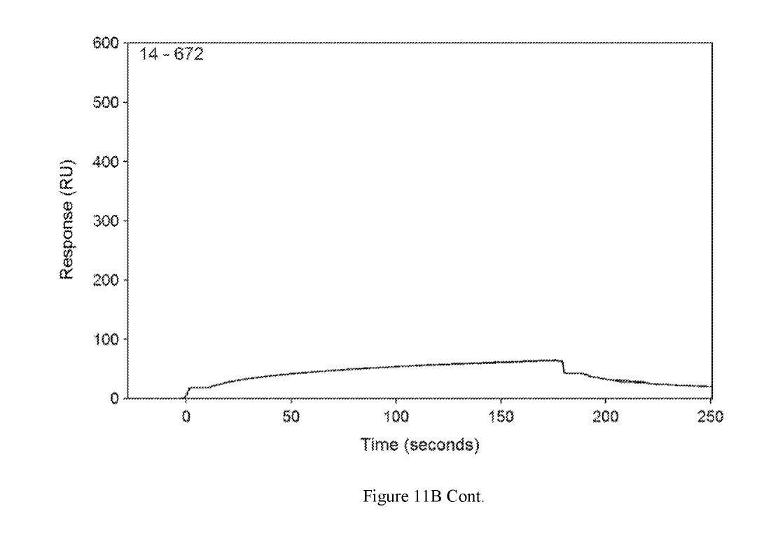

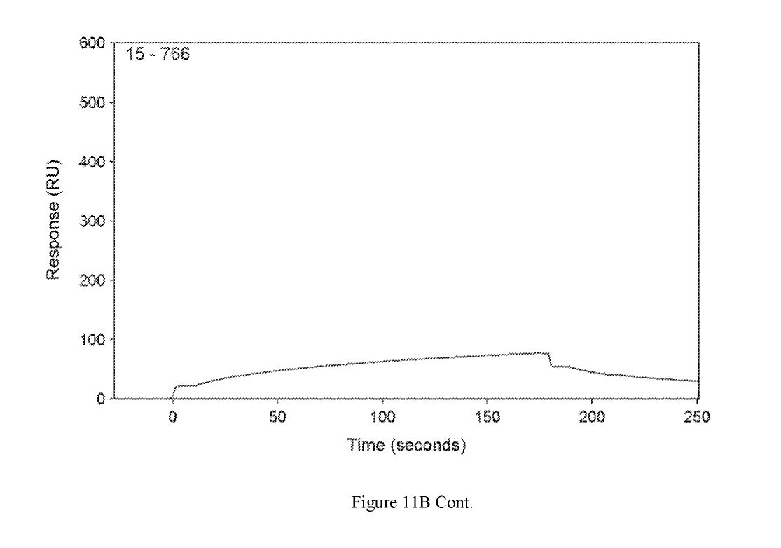

Biacore™ analysis reveals that normal human plasma contains naturally occurring antibodies that recognize both Abeta oligomers and cSNK peptides (FIG. 11), and that the binding responses are strongly correlated (FIG. 12). A further characterization of these antibodies reveals that they are mostly of the IgG1 and IgG3 subclass, with a small proportion of IgG2 and negligible or no IgG4 (FIG. 13). Finally, the strong correlation of the Biacore™ and MagPlex® immunoassays (FIG. 14), unequivocally confirms the presence of naturally occurring Abeta oligomer-specific antibodies that recognize the conformationally-constrained cSNK epitope.

### Example 8

### In Vivo Test of Passive Immunotherapies Using Hyperimmune Serum in Mouse Models

We will use the APP/PS1 and Tg2576 transgenic mice models of AD to test our hypothesis that cSNK antibody-mediated neutralization of Aβ oligomers will reduce their pathophysiologic consequences relevant to AD. Dodel et al., Naturally occurring autoantibodies against beta-amyloid: investigating their role in transgenic animal and in vitro models of Alzheimer's disease, J Neurosci, 2011, 31(15): 5847-54. Lee et al., Targeting amyloid-beta peptide (Abeta) oligomers by passive immunization with a conformation-selective monoclonal antibody improves learning and memory in Abeta precursor protein (APP) transgenic mice, J. Biol. Chem., 2006, 281(7): 4292-9. Jankowsky et al., Mutant presenilins specifically elevate the levels of the 42 residue betaamyloid peptide in vivo: evidence for augmentation of a 42-specific gamma secretase, Hum. Mol. Genet., 2004, 13(2): 159-70. Savonenko et al., Episodic-like memory deficits in the APPswe/PSIdE9 mouse model of Alzheimer's disease: relationships to beta-amyloid deposition and neurotransmitter abnormalities, Neurobiol. Dis., 2005, 18(3): 602-17. Hsiao et al., Correlative memory deficits, Abeta elevation, and amyloid plaques in transgenic mice. Science, 1996, 274(5284): 99-102.

Sex-matched cohorts of 38 mice/arm will be passively injected intraperitoneally (i.p.) to evaluate the efficacy of cSNK Aβ oligomer-based immunotherapy to prevent cognitive changes, or to slow disease progression. A cohort of old Tg2576 mice (age 14-16 months) will also be injected i.p. and compared on NOR testing against control IVIG-treated mice.

### Methods

### Passive Immunotherapies:

AAP/PS1 and Tg2576 mice will be bred and genotyped. The study will begin with 38 mice per arm in 3 arms for each mouse strain: 1) untreated, 2) IVIG enriched for antibodies specific to cSNK, 3) regular IVIG control. Mice will be housed in accredited facilities, and social enrichment will be provided.

### Antibody Therapeutic:

The two treatment arms will be delivered by intraperitoneal injection starting at 12 weeks, and once per week for the duration of the study. In a separate study, cohorts of behaviorally impaired Tg2576 mice (age 14-16 months) will also be acutely treated with IVIG enriched for antibodies specific to cSNK or regular IVIG control i.p. 1/wk×4 wks and compared on novel object recognition (NOR) testing.

### Statistics:

Power calculations are targeted for a p<0.05 statistical significance and 10% treatment effect range; we will study 19 male and 19 female mice for the possibility of sex differences in treatment response, as APP/PS1 females are known to have higher plaque burden than males [52,58]. The study includes a dropout design that will allow for neuropathological assessments in a subset of four animals at three intermediate time points, while leaving sufficient mice (N=26) for appropriately powered behavioral and neuropathological assessment at the end of the study period.

### Novel Object Recognition (NOR) Task:

The NOR task, a test for medial temporal lobe memory function in mice, and which has been extensively validated in APP/PS1 and Tg2576 mice [52, 59-63] will be used as the behavioral outcome. The time spent exploring two identical objects located in the north and south quadrant, spaced equidistant from the walls and animal is quantified. Trials are tracked using an overhead digital camera and video tracking system (Any-Maze, Stoelting, USA). 4 h after training, mice are tested by replacing one of the identical objects with a novel object of similar size. The time spent exploring the identical and novel objects is recorded and expressed as percentage of time spent exploring the novel object (duration spent with novel object/(duration spent with novel object+duration spent with familiar object)×100).

### CSF and Tissue Collection:

Animals will be anesthetized with 20 mg/kg xylazine (Bayer) and 150 mg/kg ketamine (Bimeda-MTC) via i.p. injection. CSF (approximately 15 μL/mouse) is collected from the cisterna magna as described [52,64], after which animals are transcardially perfused with PBS heparin (0.5 u/ml) for 7 min followed by tissue collection. Brains are longitudinally bisected, rapidly frozen over dry ice and stored at −80° C.

### Neuropathological and Biochemical Analysis:

One hemi-brain from each mouse will be used for histological analysis and the opposite for biochemical studies. The size, number and the area of plaques in the hippocampus and frontal cortex will be quantitated and statistically analyzed as we have described [52,64]. Immunohistochemistry will also be performed for glial fibrillary acidic protein (GFAP) and CD11b for astrocyte and microglial activation, respectively, and synaptophysin for synapse integrity. For biochemistry, brains are extracted in RIPA lysis buffer and soluble and insoluble Aβ40 and Aβ42 levels and inflammation promoting cytokines IL-6, IL-β and TNFα are measured using commercially available ELISA kits. Synaptophysin mRNA will be quantitated with RT-PCR.

### Immunological Analysis:

In addition to the inflammatory cytokines noted above, we will quantify circulating concentrations of Aβ antibodies by conventional peptide ELISA directed against Aβ oligomer-specific cSNK epitope, linearized SNK, Aβ1-40 soluble, and cell surface APP, the latter by flow cytometry. A tissue survey will also be conducted to test for reactivity of hyperimmune sera with non-target organs.

### Interpretations

It is expected that passive immunotherapies for Aβ oligomers will ameliorate behavioral deficits in APP/PS1 and Tg2576 mice, or even reverse them in acutely treated mice.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1-22. (canceled)

23. A conformational epitope-containing peptide comprising the amino acid sequence CGSNKGG (SEQ ID NO: 6), wherein said conformational epitope is in a cyclic constrained configuration.

24. The peptide of claim 23, wherein the peptide is a cyclic peptide.

25. The peptide of claim 23, wherein the peptide consists of SEQ ID NO: 6.

26. A pharmaceutical composition comprising the peptide of claim 23.

27. The pharmaceutical composition of claim 26, wherein the composition further comprises a pharmaceutically acceptable adjuvant.

28. A pharmaceutical composition comprising the peptide of claim 25.

29. The pharmaceutical composition of claim 28, wherein the composition further comprises a pharmaceutically acceptable adjuvant.

30. A method for treatment or prophylaxis of Alzheimer's disease in a subject comprising the step of administering to the subject a pharmaceutically effective amount of the pharmaceutical composition of claim 26.

31. The method of claim 30, wherein the pharmaceutical composition is administered intramuscularly, subcutaneously, intraperitoneally, orally, or with intraocular injection.

32. A method of enriching plasma for high titers of antibodies that are capable of binding an oligomeric Aβ, the method comprising immunizing an animal with the pharmaceutical composition of claim 26.

33. The method of claim 32, wherein the antibodies bind to oligomeric Aβ with greater affinity than to a non-oligomeric form of Aβ.

34. The method of claim 32, wherein the pharmaceutical composition further comprises a pharmaceutically acceptable adjuvant.

35. The method of claim 32, wherein the animal is a human.

36. The method of claim 32, wherein the method comprising the step of immunizing the animal is via intramuscular, subcutaneous, intraperitoneal or intraocular injection.

37. A method of detecting a decreased level of antibodies against oligomeric forms of Aβ(1-42) in a subject comprising the steps of:
(a) quantifying in a biological sample obtained from the subject the level of antibodies specific to a cyclic peptide comprising the amino acid sequence CGSNKGG (SEQ ID NO: 6); and
(b) comparing the level of the antibodies in step (a) with a control sample.

38. The method of claim 37, wherein the biological sample is plasma, tissues, cells, cerebrospinal fluid (CSF), blood, or combinations thereof.

39. A method for predicting a subject's risk of developing Alzheimer's Disease comprising the steps of:
(a) quantifying in a biological sample obtained from the subject the level of antibodies specific to a cyclic peptide comprising the amino acid sequence CGSNKGG (SEQ ID NO: 6); and
(b) comparing the level of the antibodies in step (a) with a control sample, wherein a lower level of the antibodies specific to the cyclic peptide compared to the control sample is indicative of the subject developing Alzheimer's Disease.

40. The method of claim 39, wherein the biological sample is plasma, tissues, cells, cerebrospinal fluid (CSF), blood, or combinations thereof.


SEQUENCE LISTING (7 sequences)

SEQ ID NO: 1 - protein, 4 aa, Artificial Sequence
  Synthetic Sequence
GSNK

SEQ ID NO: 2 - protein, 4 aa, Artificial Sequence
  Synthetic Sequence
SNKG

SEQ ID NO: 3 - protein, 5 aa, Artificial Sequence
  Synthetic Sequence
GSNKG

SEQ ID NO: 4 - protein, 5 aa, Artificial Sequence
  Synthetic Sequence
CSNKG

SEQ ID NO: 5 - protein, 7 aa, Artificial Sequence
  Synthetic Sequence
CGSNKGC

SEQ ID NO: 6 - protein, 7 aa, Artificial Sequence
  Synthetic Sequence
CGSNKGG

SEQ ID NO: 7 - protein, 8 aa, Artificial Sequence
  Synthetic Sequence
CCGSNKGC